SEQUENCE LISTING (92 sequences)

SEQ ID NO: 1 - protein, 7 aa, synthetic construct
IPIQLKA

SEQ ID NO: 2 - protein, 11 aa, synthetic construct
EEIERQLKALG

SEQ ID NO: 3 - protein, 12 aa, Homo sapiens
EDQLSRRLAALR

SEQ ID NO: 4 - protein, 12 aa, Homo sapiens
QDELSQRLARLR

SEQ ID NO: 5 - protein, 12 aa, Homo sapiens
DADLEERLKNLR

SEQ ID NO: 6 - protein, 12 aa, Homo sapiens
DEEIERQLKALG

SEQ ID NO: 7 - protein, 12 aa, Homo sapiens
LEAMQSRLATLR

SEQ ID NO: 8 - protein, 12 aa, Homo sapiens
QEELAQELLNVG

SEQ ID NO: 9 - protein, 12 aa, Homo sapiens
QEELDKNLLEIS

SEQ ID NO: 10 - protein, 12 aa, Homo sapiens
QEELNKKMTNIR

SEQ ID NO: 11 - protein, 12 aa, Homo sapiens
EDDLEAELDALG

SEQ ID NO: 12 - protein, 24 aa, Homo sapiens
PVKARPRQAELVEDAILEELSAIT

SEQ ID NO: 13 - protein, 12 aa, Homo sapiens
FDDLSRRFEELK

SEQ ID NO: 14 - protein, 24 aa, Homo sapiens
SEELEKELDILLDAELEAELEKLS

SEQ ID NO: 15 - protein, 12 aa, Saccharomyces cerevisiae
RSDTKEPLALLS

SEQ ID NO: 16 - protein, 7 aa, synthetic construct
IERQLKA

SEQ ID NO: 17 - protein, 10 aa, synthetic construct
EIERQLKALG

SEQ ID NO: 18 - protein, 12 aa, synthetic construct
DEEIERQLDALG

SEQ ID NO: 19 - protein, 7 aa, synthetic construct
IPKQEKA

SEQ ID NO: 20 - protein, 16 aa, synthetic construct
EEDDDMKELENWAGSM

SEQ ID NO: 21 - protein, 12 aa, synthetic construct
DEELERRLKALK

SEQ ID NO: 22 - protein, 16 aa, synthetic construct
EQDELSQRLARLRDQV

SEQ ID NO: 23 - protein, 16 aa, synthetic construct
VPVKARPRQAELVAAS

SEQ ID NO: 24 - protein, 10 aa, synthetic construct
RNERQLKALG

SEQ ID NO: 25 - protein, 12 aa, synthetic construct
EEEIVRQLKALG

SEQ ID NO: 26 - protein, 12 aa, synthetic construct
DIEIEFQLKALG

SEQ ID NO: 27 - protein, 10 aa, synthetic construct
EIERQLKAQI

SEQ ID NO: 28 - protein, 12 aa, synthetic construct
DEEYERQWKALG

SEQ ID NO: 29 - protein, 12 aa, synthetic construct
DEAIERVLKALG

SEQ ID NO: 30 - protein, 12 aa, synthetic construct
DDEIEVQLKALG

SEQ ID NO: 31 - protein, 10 aa, synthetic construct
TLEIERQLKA

SEQ ID NO: 32 - protein, 12 aa, synthetic construct
LEEIERQLKALG

SEQ ID NO: 33 - protein, 18 aa, synthetic construct
KEEEDDDMKELENWAGSM

SEQ ID NO: 34 - protein, 12 aa, synthetic construct
NEEGAPQEGILE

SEQ ID NO: 35 - protein, 12 aa, synthetic construct
DEEYCRQWKALC

SEQ ID NO: 36 - protein, 12 aa, synthetic construct
DIEICFQLKALC

SEQ ID NO: 37 - protein, 12 aa, synthetic construct
DEEICRQLDALC

SEQ ID NO: 38 - protein, 31 aa, synthetic construct
TFFYGGSRGKRNNFKTEEYDEEICRQLDALC

SEQ ID NO: 39 - protein, 31 aa, synthetic construct
TFFYGGSRGKRNNFKTEEYGEARCEIQDLLC

SEQ ID NO: 40 - protein, 30 aa, synthetic construct
FFYGGSRGKRNNFKTEEYDEEIERQLDALG

SEQ ID NO: 41 - protein, 31 aa, synthetic construct
TFFYGGSRGKRNNFKTEEYGEARDEIQDLLE

SEQ ID NO: 42 - DNA, 52 bp, synthetic construct
  source 1..52: mol_type = other DNA
aatgatacggcgaccaccgaaatggactatcatatgcttaccgtaacttgaa

SEQ ID NO: 43 - DNA, 53 bp, synthetic construct
  source 1..53: mol_type = other DNA
caagcagaagacggcatacgatgtggatgaatactgccatttgtctcgaggtc

SEQ ID NO: 44 - DNA, 36 bp, synthetic construct
  source 1..36: mol_type = other DNA
catccgctagcggatcccatggccagtggaatcacc

SEQ ID NO: 45 - DNA, 45 bp, synthetic construct
  source 1..45: mol_type = other DNA
ccataccggtagatgtatactaatgaaggtaatagcttgaaagag

SEQ ID NO: 46 - DNA, 34 bp, synthetic construct
  source 1..34: mol_type = other DNA
gttgaccggtatggtgtctaagggcgaagagctg

SEQ ID NO: 47 - DNA, 39 bp, synthetic construct
  source 1..39: mol_type = other DNA
ggcagaattcgaattaagtttgtgccccagtttgctagg

SEQ ID NO: 48 - protein, 12 aa, synthetic construct
FEELEAQLARLR

SEQ ID NO: 49 - protein, 7 aa, synthetic construct
RAPSCHL

SEQ ID NO: 50 - protein, 7 aa, synthetic construct
DWLMVNL

SEQ ID NO: 51 - protein, 7 aa, synthetic construct
LPASMPC

SEQ ID NO: 52 - protein, 7 aa, synthetic construct
LQVGVAV

SEQ ID NO: 53 - protein, 7 aa, synthetic construct
PLPTGMG

SEQ ID NO: 54 - protein, 7 aa, synthetic construct
CMVHSAG

SEQ ID NO: 55 - protein, 7 aa, synthetic construct
AAYNLCA

SEQ ID NO: 56 - protein, 7 aa, synthetic construct
LSGECVP

SEQ ID NO: 57 - protein, 7 aa, synthetic construct
GAAAGGT

SEQ ID NO: 58 - protein, 7 aa, synthetic construct
QSVVTAP

SEQ ID NO: 59 - protein, 7 aa, synthetic construct
CKYRSVL

SEQ ID NO: 60 - protein, 7 aa, synthetic construct
PAMQIDG

SEQ ID NO: 61 - protein, 7 aa, synthetic construct
GWTTVRR

SEQ ID NO: 62 - protein, 7 aa, synthetic construct
QSVLQQV

SEQ ID NO: 63 - protein, 7 aa, synthetic construct
QSPSSAW

SEQ ID NO: 64 - protein, 7 aa, synthetic construct
QVSVAQQ

SEQ ID NO: 65 - protein, 7 aa, synthetic construct
QAELSNS

SEQ ID NO: 66 - protein, 7 aa, synthetic construct
EDVNKCV

SEQ ID NO: 67 - protein, 7 aa, synthetic construct
WPRYPHI

SEQ ID NO: 68 - protein, 7 aa, synthetic construct
CFLFQIQ

SEQ ID NO: 69 - protein, 7 aa, synthetic construct
ECHTKIR

SEQ ID NO: 70 - protein, 7 aa, synthetic construct
MQMKLSQ

SEQ ID NO: 71 - protein, 7 aa, synthetic construct
TQKRELT

SEQ ID NO: 72 - protein, 7 aa, synthetic construct
VYLDILG

SEQ ID NO: 73 - protein, 7 aa, synthetic construct
LKRDQPV

SEQ ID NO: 74 - protein, 7 aa, synthetic construct
NNQYSFV

SEQ ID NO: 75 - protein, 7 aa, synthetic construct
LPSKIYK

SEQ ID NO: 76 - protein, 12 aa, Homo sapiens
DEEALKQLAEWV

SEQ ID NO: 77 - protein, 12 aa, Homo sapiens
EDDDMKELENWA

SEQ ID NO: 78 - protein, 12 aa, Homo sapiens
EDDDIKQLAAWA

SEQ ID NO: 79 - protein, 12 aa, Homo sapiens
EDAILEELSAIT

SEQ ID NO: 80 - protein, 12 aa, Saccharomyces cerevisiae
EDEDEKALRELQ

SEQ ID NO: 81 - protein, 12 aa, Saccharomyces cerevisiae
VNEMRERLRALQ

SEQ ID NO: 82 - protein, 12 aa, Saccharomyces cerevisiae
DDDLQARLNTLK

SEQ ID NO: 83 - protein, 12 aa, Saccharomyces cerevisiae
EDKLAQRLRALR

SEQ ID NO: 84 - protein, 4 aa, synthetic construct
DEEI

SEQ ID NO: 85 - protein, 15 aa, synthetic construct
ATVAEKTKEQVTNVG

SEQ ID NO: 86 - protein, 12 aa, synthetic construct
AGKTKEGVLYVG

SEQ ID NO: 87 - protein, 12 aa, synthetic construct
AATGFVKKDQLG

SEQ ID NO: 88 - protein, 12 aa, synthetic construct
MDVFMKGLSKAK

SEQ ID NO: 89 - protein, 7 aa, synthetic construct
RPIQLIA

SEQ ID NO: 90 - protein, 12 aa, synthetic construct
EDELEKRLAALR

SEQ ID NO: 91 - protein, 12 aa, Saccharomyces cerevisiae
DAELDAELDALA

SEQ ID NO: 92 - protein, 12 aa, Saccharomyces cerevisiae
LDELKKRFDALR

(19) United States
(12) Patent Application Publication
(10) Pub. No.: US 2025/0051391 A1
(43) Pub. Date: Feb. 13, 2025

(54) PEPTIDE INHIBITORS AND METHODS FOR INHIBITING PROTEIN AGGREGATION IN NEURONS AND NEURODEGENERATIVE DISEASES

(71) Applicants: THE GOVERNING COUNCIL OF THE UNIVERSITY OF TORONTO, Toronto (CA); UNIVERSITY HEALTH NETWORK, Toronto (CA)

(72) Inventors: Philip Kim, York (CA); Satra Nim, Toronto (CA); Carlos Corbi Verge, Etobicoke (CA); Suneil K. Kalia, Toronto (CA); Lorraine V. Kalia, Toronto (CA)

(21) Appl. No.: 18/720,385

(22) PCT Filed: Dec. 15, 2022
(86) PCT No.: PCT/CA2022/051837

§ 371 Date: Jun. 14, 2024

Related U.S. Application Data
(60) Provisional application No. 63/289,912, filed on Dec. 15, 2021.

(51) Int. Cl. C07K 7/06; A61K 38/00; A61P 25/28; C07K 7/08

(52) U.S. Cl. CPC C07K 7/06; A61P 25/28; C07K 7/08; A61K 38/00

(57) ABSTRACT

Provided herein is a method of decreasing a-syn levels and/or decreasing a-syn toxicity in a cell, the method comprising contacting the cell with a charged multivesicular body protein 2B: a-synuclein (CHMP2B:a-syn) inhibitor and a method of inhibiting neural degeneration, the method comprising administering to a subject in need thereof a charged multivesicular body protein 2B: a-synuclein (CHMP2B:a-syn) inhibitor.

### CROSS-REFERENCE TO RELATED APPLICATIONS

This application claims the benefit of priority to U.S. Provisional Application No. 63/289,912, filed Dec. 15, 2021, the contents of which is incorporated herein by reference in its entirety.

### INCORPORATION OF SEQUENCE LISTING

A computer readable form of the Sequence Listing “2223-P65207PC00” (81, 020 bytes) created on Dec. 15, 2022, is herein incorporated by reference.

### FIELD

The present disclosure relates to peptide inhibitors that reduce a-synuclein toxicity through binding with CHMPB2 and/or inhibiting the interaction between CHMPB2 and a-synuclein and their use in treating synucleopathies.

### BACKGROUND

Protein-protein interactions (PPIs) govern virtually all molecular pathways involved in cell growth, differentiation, and survival (1, 2). Inhibition of PPIs with peptides or small molecules to modulate these pathways has proven to be successful for the treatment of cancers (3, 4). PPI inhibitors could conceivably be a promising new therapeutic venue for neurodegenerative proteinopathies, such as Parkinson's disease (PD), for which no disease-modifying therapies exist (5). Wild-type (WT) or mutant a-synuclein protein (a-syn) accumulates in PD to form oligomers that disrupt core cellular systems causing neurodegeneration (6). Rescuing these toxic effects by targeting PPIs has been an unexplored therapeutic strategy for PD (7, 8). Identification of a-syn sequence based putative PPI inhibitors that reduce a-syn accumulation have been described (9, 59).

### SUMMARY

A first aspect is directed to a method of decreasing a-syn levels and/or decreasing a-syn toxicity in a cell, the method comprising contacting the cell with a charged multivesicular body protein 2B: a-synuclein (CHMP2B:a-syn) inhibitor.

Another aspect is directed to a method of inhibiting neural degeneration, the method comprising administering to a subject in need thereof a charged multivesicular body protein 2B: a-synuclein (CHMP2B:a-syn) inhibitor.

Another aspect is directed to a charged multivesicular body protein 2B: a-synuclein (CHMP2B:a-syn) inhibitor comprising a peptide comprising i) a MIT-Interacting Motif (MIM) sequence or a sequence with at least 50% sequence identity to said MIM sequence; or 2) an a-syn interaction sequence or a sequence with at least 50% sequence identity to said a-syn interaction sequence, wherein the peptide inhibits CHMP2B-a-syn interaction by at least 50%.

A further aspect is directed to a peptide, the peptide consisting of 5 to 213 amino acids, preferably, 5 to 25 amino acids, 5 to 30 amino acids, or 7 to 30 amino acids, and comprising i) a MIT-Interacting Motif (MIM) sequence or a sequence with at least 50% sequence identity to said MIM sequence; or 2) an a-syn interaction sequence or a sequence with at least 50% sequence identity to said a-syn interaction sequence, wherein the peptide inhibits CHMP2B-a-syn interaction by at least 50%.

Yet another aspect is directed to a nucleic acid molecule comprising a polynucleotide sequence encoding any peptide or polypeptide described herein.

Another aspect is directed to a vector comprising a vector backbone and any nucleic acid molecule described herein.

A further aspect is directed to a recombinant cell recombinantly expressing any peptide or polypeptide, any nucleic acid molecule or any vector described herein.

Another aspect is directed to a composition comprising any peptide, polypeptide, CHMP2B:a-syn inhibitor, nucleic acid, vector or recombinant cell described herein.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIGS. 1A-F show Proteomic screens and in vitro validation of hits. (A) Schematic of the experimental design including proteomic screens, identification and validation of hits, target deconvolution, and evaluation in cell and animal models. For the cytotoxicity screen, peptide library to identify peptides that rescue cytotoxicity induced by a-syn overexpression and oligomers was employed. To screen the library for peptides that inhibit a-syn oligomers, FACS with a split YFP-a-syn system (cells co-expressing V1S and SV2) was used. (B) Validation of cell viability effect of peptides in A53T and WT a-syn expressing cells under proteostatic stress (due to MG132 administration) as well as controls. Experiments were done in triplicate. Data represent mean values±s.d. (C) WT a-syn oligomers as measured by luciferase activity. All 4 peptides showed significant reduction in a-syn oligomers from cells stably expressing split luciferase a-syn constructs. Experiments were done in triplicate. Data represent mean values±s.d. (D) Co-immunoprecipitation experiment with Flag-CHMP2B and GFP-peptides. PDpep1 and PDpep1.3 peptides interacted with Flag-CHMP2B whereas GFP alone (CTL) did not. (E) Fluorescence polarization (FP) binding assay of a FITC-labeled PDpep1.3 peptide against CHMP2B. Error bars represent ±s.d. of the fit with n=5. (F) shRNA-induced cell viability rescue experiment. Cell viabilities were measured with cells stably expressing different shRNAs and transfected with Scramble or PDpep1.3 peptide. Experiments were done in triplicate. Data represent mean values±s.d. **P<0.01; ***P<0.001; ****P<0.0001.

FIGS. 2A-L show PDpep1.3 reduces a-syn levels in cell lines and primary cortical neurons. (A) Representative immunoblots of a-syn levels in HEK293 cells stably overexpressing A53T a-syn and infected with different peptides or full-length CHMP2B (top panel), and beta-actin levels as control (middle panel). RT-PCR shows no change in mRNA levels in HEK293 cells stably expressing A53T a-syn (bottom panel). (B) Quantification of A53T protein expression level from immunoblot data in (A). Student t-test (n=2). (C) Quantification of a-syn transcript level from RT-PCR data in (A). (D) Representative images of rat primary cortical neurons transduced with A53T a-syn plus Scramble1.3-GFP or PDpep1.3-GFP (scale bars=5 μm). (E) Quantification of relative a-syn fluorescence in GFP positive neurons. Nested t-test (n=3). (F) Representative immunoblot of GFP (top panel) and human a-syn (bottom panel) of lysates from primary cortical neurons transduced with A53T a-syn plus Scramble1.3-GFP or PDpep1.3-GFP. (G) Quantification of relative endogenous a-syn fluorescence in GFP positive neurons. Nested t-test (n=3). (H) Representative images of rat primary cortical neurons transduced with V1S/SV2 or YFP plus Scramble1.3-RFP or PDpep1.3-RFP to assess for a-syn oligomer levels (YFP). Quantification of relative a-syn fluorescence in RFP positive neurons in primary cortical neurons transduced with V1S/SV2 plus Scramble1.3-RFP or PDpep1.3-RFP (I) or V1S alone plus Scramble1.3-RFP or PDpep1.3-RFP (J). Nested t-test (n=3). Quantification of relative YFP fluorescence in RFP positive neurons in primary cortical neurons transduced with V1S/SV2 plus Scramble1.3-RFP or PDpep1.3-RFP (K) or YFP plus Scramble1.3-RFP or PDpep1.3-RFP (L). Nested t-test (n=3). *P<0.05; **P<0.01; ****P<0.0001; ns indicates P>0.05.

FIGS. 3A-J show PDpep1.3 outcompetes a-syn binding to CHMP2B to enhance lysosome-mediated clearance of a-syn. (A) Validation of PPI disruption using co-immunoprecipitation assays. Flag-CHMP2B was immunoprecipitated in the presence of HA-tagged A53T a-syn and GFP-tagged peptides. For controls, GFP alone (CTL) and a GFP-tagged scrambled version of the initial peptide (Scramble1.3) were included; neither co-immunoprecipitated with CHMP2B nor disrupted the interaction between CHMP2B and A53T a-syn. (B) FP binding assay of FITC-labeled PDpep1.3 and displacement of the fluorescent peptide with increasing concentrations of a-syn. Error bars represent ±s.d. of the fit with n=3 (left panel). FP binding assay of a collection of FITC-labelled a-syn peptides. Error bars represent ±s.d. of the fit with n=2 (right panel). SEQ ID NOs: 6, 34, and 85-88 are included. (C) PDpep1.3 restores reduced LAMP1 expression by A53T a-syn as shown by confocal micrographs taken 48 h after co-transfection of A53T and/or the indicated peptide in HEK293 cells (scale bars=15 μm). (D) Representative immunoblots of LAMP1 protein levels in HEK293 cells upon transient overexpression of A53T and/or peptides 48 h after co-transfection using an anti-LAMP1 antibody. Controls were GFP alone (pLJM1) and a GFP-tagged scrambled version of the initial peptide (scramble). Loading control was beta-actin. Experiments were done in triplicate. (E) Representative images of primary cortical neurons transduced with A53T a-syn or empty vector (EV) plus Scramble1.3-RFP or PDpep1.3-RFP (scale bars=5 μm). (F) Quantification of LAMP1 fluorescence in RFP positive neurons. Nested one-way ANOVA (n=3). (G) Representative immunoblots of CD63 protein levels in HEK293 cells upon transient overexpression of A53T and/or peptides 48 h after co-transfection using an anti-CD63 antibody. Controls were GFP alone (pLJM1) and a GFP-tagged scrambled version of the initial peptide (scramble). Loading control was beta-actin. Experiments were done in triplicate. (H) Lysosome activity assay using flow cytometry in HEK293 cells co-transfected with A53T a-syn or EV plus Scramble1.3 or PDpep1.3. Cells were treated with the lysosomal inhibitor Leupeptin (Leu) as indicated. Experiments were done in duplicate. Data represent mean values ±s.d. (I) shRNA-induced cell viability rescue experiment. Cell viabilities in HEK293 cells were measured with cells stably expressing p62 shRNA and transfected with GFP alone (pLJM1) or with GFP-tagged scramble or PDpep1.3 peptide. Experiments were done in triplicate. Data represent mean values ±s.d. (J) Proposed mechanism of PDpep1.3 in which the peptide disrupts the a-syn-CHMP2B interaction to restore lysosomal degradation of a-syn. *P<0.05; **P<0.01.

FIGS. 4A-J show PDpep1.3 reduces a-syn-mediated neurodegeneration in C. elegans and an a-syn oligomer rat model. (A) Representative image of an adult C. elegans with PDE neuron visualized using dat-1p::gfp (eg/s1) (scale bar=20 μm) (top panel). Neurite length for each PDE neuron was categorized as: Short (neurite does not extend past the vulva; purple), Medium (neurite extends past the vulva but not beyond halfway to ADE neuron; cyan), or Long (neurite extends to ADE neuron). (B) Neurite length of PDE neurons for C. elegans expressing no a-syn (No a-syn), a-syn alone (a-syn), a-syn with or without RFP (a-syn;TagRFP Marker+ or a-syn;TagRFP Marker-, respectively), or a-syn with or without RFP-PDpep1.3 (a-syn;TagRFP::PDpep1.3 Marker+ or a-syn;TagRFP::PDpep1.3 Marker-, respectively). Pearson Chi-Square test for pairwise comparison of frequencies between two populations. (n=7 to 15 plates of worms/group). (C) Representative images of native YFP and RFP fluorescence and immunofluorescent staining with anti-tyrosine hydroxylase (TH) antibody in SN of rats injected with V1S/SV2 or YFP plus Scramble1.3-RFP or PDpep1.3-RFP (scale bars=200 μm). Quantification of YFP⁺ area in SN of rats injected with V1S/SV2 (D) and full length YFP (E). Quantification of YFP⁺ area in striatum of rats injected with V1S/SV2 (F) and full length YFP (G). Quantification of a-syn⁺ area in SN (H), TH⁺ cell counts in SN (I), and TH fluorescence in striatum (J) at 6 weeks post injection of V1S/SV2 plus Scramble1.3-RFP or PDpep1.3-RFP. Bars represent means±s.e.m. Unpaired t-test (n=7 to 10 rats/group). *P<0.05; **P<0.01; ***P<0.001; ****P<0.0001; ns indicates P>0.05.

FIGS. 5A-K show PDpep1.3 reduces a-syn levels and a-syn-mediated neurodegeneration in a preclinical rat model of PD. (A) Experimental design and timeline for testing PDpep1.3 versus Scramble1.3 in AAV vector-based A53T a-syn rat model. (B) Representative images of immunofluorescent staining (with anti-a-syn or anti-TH antibodies) and native GFP fluorescence in SN of rats injected with low titer A53T a-syn or empty vector (EV) plus Scramble1.3-GFP or PDpep1.3-GFP (scale bars=200 μm). TH⁺ cell counts (C), quantification of a-syn⁺ area (D), and LAMP1⁺ puncta counts (E) in SN at 6 weeks post injection of low titer A53T a-syn or EV plus Scramble1.3-GFP or PDpep1.3-GFP. Bars are means±s.e.m. One-way ANOVA followed by Dunnett's post-test (n=5 to 7 rats/group). (F) Representative images of immunofluorescent staining (with anti-a-syn or anti-TH antibodies) and native GFP fluorescence in SN of rats injected with high titer A53T a-syn or EV plus Scramble1.3-GFP or PDpep1.3-GFP (scale bars=200 μm). (G) TH⁺ cell counts in SN at 6 weeks post injection of high titer A53T a-syn or EV plus Scramble1.3-GFP or PDpep1.3-GFP. (H) Forelimb asymmetry in cylinder test at baseline (prior to injection), 3 weeks post injection (WPI), and 6 WPI. Data are mean±s.e.m. Repeated measures ANOVA (n=6 to 8 rats/group). Quantification of dopamine (I), DOPAC (J), and HVA (K) in striatum. For G, I, J, K, bars are means±s.e.m. One-way ANOVA followed by Dunnett's post-test (n=7 to 8 rats/group). *P<0.05; **P<0.01; ****P<0.0001; ns indicates P>0.05.

FIGS. 6A-F show PDpep1.3 reduces endogenous a-syn levels and rescues lysosomal activity in human PD cell models. (A) Representative images of fibroblasts from a PD patient with SNCA triplication transduced with Scramble1.3-RFP or PDpep1.3-RFP (scale bars=10 μm). (B) Quantification of relative a-syn fluorescence in RFP positive fibroblasts. Nested t-test (n=3). (C) Lysosome activity assay using confocal microscopy in SNCA triplication fibroblasts transduced with AAV-Scramble1.3-RFP or AAV-PDpep1.3-RFP. Cells were treated with the lysosomal inhibitor Bafilomycin A1 (Baf) as indicated. Nested one-way ANOVA with Dunnett's post-test (n=3). (D) Representative images of human iPSC-derived dopaminergic neurons with A53T a-syn mutation or isogenic controls without a-syn mutation. These iPSC-derived dopaminergic neurons were transduced with Scramble1.3-RFP or PDpep1.3-RFP and immunostained with anti-a-syn, anti-TH, or anti-LAMP1 antibodies (scale bars=5 μm). (E) Quantification of relative a-syn fluorescence intensity in RFP positive A53T mutant and control iPSC-derived dopaminergic neurons. Nested t-tests (n=3). (F) Quantification of relative LAMP1 fluorescence intensity in RFP positive A53T mutant and control iPSC-derived dopaminergic neurons. Nested t-tests (n=3). *P<0.05; **P<0.01; ****P<0.0001; ns indicates P>0.05.

FIGS. 7A-C show Peptides are cytoprotective in the absence of MG132, PDpep1.3 disrupts CHMP2B-VPS4 interaction, and shRNA constructs knock down gene expression. (A) Validation of cell viability effect of peptides in A53T or WT-a-syn expressing cells without the addition of MG132. Experiments were done in triplicate. Data represent mean values±s.d. (B) Validation of PPI disruption using co-immunoprecipitation assays. Flag-VPS4B was immunoprecipitated in the presence of different HA-tagged CHMP proteins and GFP-tagged peptides. PDpep1.3 disrupted the interaction between VPS4B and CHMP2B. GFP was used alone as a control (CTL). (C) Reduction in gene expression level with shRNA knockdown. Expression of targeted gene was measured in cells stably expressing shRNA as indicated. Experiments were done in triplicate. Data represent mean values±s.d. **P<0.01; ***P<0.001.

FIG. 8 shows PDpep1.3 interacts with CHMP2B and VPS4B. Fluorescence polarization (FP) binding assay of a FITC-labeled DEEIERQLKALG (SEQ ID NO: 6) peptide against CHMP2B (Kd=1.8 μM) and VPS4B (Kd≈200 μM).

FIGS. 9A-B show differences of PDpep1.3 to other MIMs in sequence (alignment) and in cytoprotective effects. (A) Overlapping sequences of different MIMs as displayed in Ugene (14) and aligned with ClustalW (15). CHMP1B-MIM represents a classic MIM and CHMP2B-super is a modified version of PDpep1.3 that makes it closer to a classic MIM. SEQ ID NOs: 1, 16, 19, 17, 6, 20-23, 3, 8, 7, 13 and 89 are included. (B) Validation of cell viability effect of MIM motif peptides in A53T expressing cells as well as controls. GFP was used alone as a control (CTL). Experiments were done in triplicate. Data represent mean values±s.d. **P<0.01; ***P<0.001; ****P<0.0001.

FIGS. 10A-F show Structure of a-syn oligomer with PDpep1.3 and effects of truncated CHMP2B. (A) Structure of a-syn and binding region to CHMP2B (in violet). (B) Schematic of CHMP2B truncations. PDpep1, PDpep1.3, and CHMP2B 1-199 truncation are each associated with a reduction in a-syn levels by immunoblot. Quantification of a-syn levels is shown in the bar graph. Experiments were done in duplicate. Data represent mean values±s.d. (C) Cell viability assay in A53T cell line with peptides and truncated CHMP2B proteins. Experiments were done in triplicate. Data represent mean values±s.d. (D) a-syn mutations affect LAMP1 expression (left) and cell viability (right). Experiments were done in triplicate. Data represent mean values±s.d. (E) Representative images of rat primary cortical neurons transduced with a truncated version of A53T a-syn lacking amino acids 103-140 (Aa-syn (103-140)) which was HA-tagged plus Scramble1.3-GFP or PDpep1.3-GFP (scale bars=5 μm). (F) Quantification of relative Δa-syn(103-140) fluorescence (by immunostaining with anti-HA antibodies) in GFP positive neurons. Nested t-test (n=3). *P<0.05; **P<0.01; ns indicates P>0.05.

FIGS. 11A-B show PDpep1.3 rescues TH levels in striatum and increases LAMP1 levels in SN. (A) Representative images showing immunofluorescent staining with anti-tyrosine hydroxylase (TH) antibody and native fluorescence (YFP or RFP) in the striatum of rats injected with V1S/SV2 or YFP plus Scramble1.3-RFP or PDpep1.3-RFP (scale bars=500 μm). (B) Representative z-stack projection images showing immunofluorescent staining (with anti-LAMP1 or anti-a-syn antibodies) and native GFP fluorescence in SN of rats injected with low-titer A53T a-syn plus Scramble-GFP or PDpep1.3GFP (top two panels); or low-titer EV plus Scramble-GFP or PDpep1.3-GFP (bottom two panels) (scale bars=10 μm).

FIGS. 12-A-C show (A) Selection of top 10 peptides from PD Optimization Library. Parent, WT asyn and A53T cell lines were used to screen for effective peptides that rescue cell viability from toxic expression of alpha-synuclein. (B) Peptide-induced cell viability rescue experiment. Cell viabilities in A53T cells were measured by transfecting different concentrations of the different versions of the optimized PDpep1.3 peptide, the scramble peptide or PDpep1.3 peptide. (C) Peptide-induced cell viability rescue experiment. Cell viabilities in A53T cells were measured by transfecting the highest concentration (0.2 ug) of the different versions of the optimized PDpep1.3 peptide, the scramble peptide or PDpep1.3 peptide.

FIGS. 13A-B show (A) Peptide-induced cell viability rescue experiment. Cell viabilities in A53T cells were measured by incubating different concentrations of the stapled peptides. SEQ ID NOs: 18, 24-32 are included. (B) WT a-syn aggregation as measured by luciferase activity. Stapled peptides DEEYCRQWKALC (SEQ ID NO: 35), DIEICFQLKALC (SEQ ID NO: 36) and DEEICRQLDALC (SEQ ID NO: 37) showed significant reduction in a-syn aggregation from cells stably expressing split luciferase a-synuclein constructs.

FIG. 14 shows the results of the fluorescence polarization (FP) binding assay of a FITC-labeled PDpep1.3 peptide against CHMP2B 1-199 truncation and CHMP2B full length. Error bars represent ±s.d of the fit with n=5. SEQ ID NOs: 36-37 and 84 are included.

FIG. 15 depicts sequences of different MIMs as displayed in Ugene (14) and aligned with ClustalW (15). The sequence at the top of the figure shows amino acids in capital letters that are much more conserved than others. Amino acids in lowercase letters are slightly conserved but less conserved than amino acids in capital letters. SEQ ID NOs: 90, 3-7, 76-79, 11, 13-15, 80-83, and 91-92 are included.

FIG. 16 is a graph depicting validation of cell viability effect of peptides in A53T expressing HEK293 cells at different concentrations of cyclized and non-cyclized angio-conjugated peptides and cyclic bisphenyl conjugated peptides.

### DETAILED DESCRIPTION OF THE DISCLOSURE

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

In understanding the scope of the present disclosure, the term “comprising” and its derivatives, (such as “comprise” and “comprises”), “having” (and any form of having, such as “have” and “has”), “including” (and any form of including, such as “include” and “includes”) or “containing” (and any form of containing, such as “contain” and “contains”), as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”. For ranges described herein, subranges are also contemplated, for example every, 0.1 increment there between. For example, if the range is 80% to about 90%, also contemplated are 80.1% to about 90%, 80% to about 89.9%, 80.1% to about 89.9% and the like.

The term “cell” as used herein refers to a single cell or a plurality of cells.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue or non-natural amino acid in place of a non-derivatized residue provided that such polypeptide displays the requisite activity.

As used herein, the terms “peptide,” “polypeptide,” and “protein” refer to any chain of two or more natural or unnatural amino acid residues, regardless of post-translational modifications (e.g., glycosylation or phosphorylation). The polypeptides incorporated into the biphasic vesicles of the disclosure can include for example from 3 to 3500 natural or unnatural amino acid residues. Included are proteins that are a single polypeptide chain and multisubunit proteins (e.g. composed of 2 or more polypeptides).

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecule of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The terms “nucleic acid” or “oligonucleotide” as used herein means two or more covalently linked nucleotides. Unless the context clearly indicates otherwise, the term generally includes, but is not limited to, deoxyribonucleic acid (DNA) and ribonucleic acid (RNA), which may be single-stranded (ss) or double stranded (ds). For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The term “oligonucleotide” as used herein generally refers to nucleic acids up to 200 base pairs in length and may be single-stranded or double-stranded. The sequences provided herein may be DNA sequences or RNA sequences, however it is to be understood that the provided sequences encompass both DNA and RNA, as well as the complementary RNA and DNA sequences, unless the context clearly indicates otherwise. For example, the sequence 5′-GAATCC-3′, is understood to include 5′-GAAUCC-3′, 5′-GGATTC-3′, and 5′GGAUUC-3′.

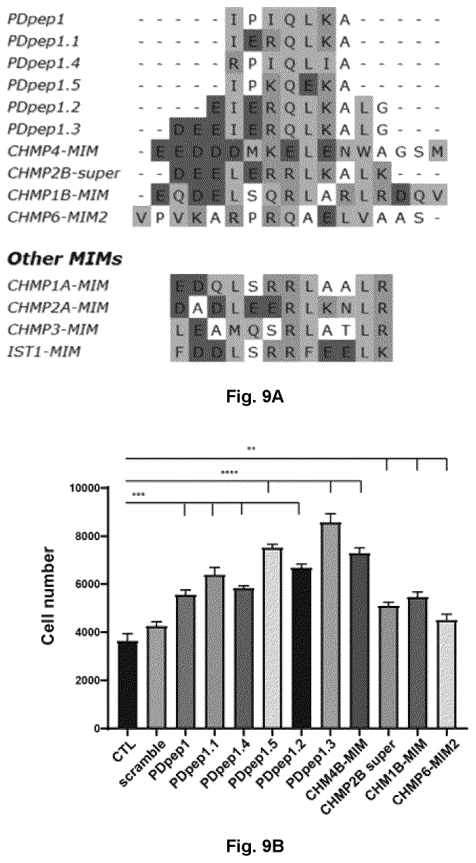

The term “MIT-Interacting Motif (MIM) sequence” as used herein means a sequence that has been identified as such including those in FIG. 9, and fragments thereof of that are at least 5 amino acids long and that inhibit CHMP2B a-syn interaction by at least 50% (which can be referred to as biologically active fragments). The sequences can be mammalian such as human and may be wildtype or may comprise one or more mutations. Decreased interaction can be assessed by comparing interaction in the presence or absence of the MIM sequence or fragment, or by comparing interaction in the presence of the MIM sequence or fragment compared to a control peptide such as a scrambled peptide.

The term “a-syn interaction sequence” as used herein means an a-syn sequence comprising amino acids 103-114 (Accession number NM_000345.4) or a fragment thereof that is at least 5 amino acids long or at least 7 amino acids long (for example amino acids 103-109, 104-110, 105-111, 106-112, 107-1113 or 108-114, 103-110, 104-111, 105-112 . . . 103-111, 104 to 112 etc.) and that inhibit CHMP2B a-syn interaction by at least 50% (which can be referred to as biologically active fragments). The sequences can be mammalian such as human, and may be wildtype as for example as provided in. Decreased interaction can be assessed by comparing interaction in the presence or absence of the a-syn sequence or fragment, or by comparing interaction in the presence of the a-syn sequence or fragment compared to a control peptide such as a scrambled peptide.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods and Compositions

Accumulation of a-syn into toxic oligomers mediates dopaminergic neurodegeneration in Parkinson's disease and other synucleinopathies. A high-throughput, proteome-wide screen was performed as described in the Examples which identified protein-protein interaction inhibitors that reduced a-syn oligomer formation and rescued a-synuclein toxicity. One of the peptide inhibitors disrupted a previously unknown interaction between the C-terminal region of a-synuclein and charged multivesicular body protein 2B (CHMP2B), a component of the endosomal sorting complex required for transport III (ESCRT-III). It is shown that, through this interaction, a-synuclein disrupts lysosomal activity thereby inhibiting its own degradation; this effect may be amplified upon a-syn oligomerization. Conversely, the peptide inhibitor restored lysosomal activity and thus led to decrease of a-syn levels, rescuing a-syn toxicity. It also lowered a-syn levels in preclinical rodent models of Parkinson's disease and in human cells harboring disease-causing a-synuclein mutations, including iPSC-derived dopaminergic neurons. Furthermore, the peptide inhibitor protected dopaminergic neurons from a-syn-mediated degeneration in C. elegans and preclinical rodent models of Parkinson's disease.

Accordingly a first aspect relates to a method of decreasing a-syn levels and/or decreasing a-syn toxicity in a cell, the method comprising contacting the cell with a charged multivesicular body protein 2B: a-synuclein (CHMP2B:a-syn) inhibitor.

As demonstrated herein, peptide inhibitors were identified that decreased a-syn levels and decreased a-syn toxicity in vitro and in vivo. As demonstrated in the Examples, a peptide comprising the amino acid sequence IPIQLKA (SEQ ID NO: 1) was identified as inhibitory in the screen and corresponds to residues 203-209 in uniprot ID: Q9UQN3. The inhibitory peptide identified in the screen mapped to the C-terminal domain of charged multivesicular body protein 2B (CHMP2B) MIT binding motif (MIM). The MIM has the sequence of EEIERQLKALG (SEQ ID NO: 2) and corresponds to residues 201-211 in uniprot ID: Q9UQN3.

Additional peptide inhibitors were identified and tested.

For example, different peptides based or related to the identified sequence or a MIM domain, for example from other CHMP proteins, were also able to inhibit interaction with a-syn or CHMP self interaction by at least 50%.

Accordingly in an embodiment, the CHMP2B:a-syn inhibitor is or comprises a peptide.

In an embodiment, the peptide is or comprises a CHMP2B MIM sequence.

The a-syn interaction domain with CHMP2B was also mapped.

In another embodiment, the peptide is or comprises an a-syn interaction sequence.

Various peptides can be used based on, for example, known MIM and a-syn sequences, variations thereof and sequences described herein. In an embodiment, the peptide is one that inhibits interaction of CHMP2B and a-syn or CHMP2B self interaction by at least 50%, at least 60%, at least 70% or at least 80%.

Inhibition of the interaction can be assessed for example by a competition assay, for example as described in the Examples. In some embodiments, the inhibition of the interaction can be assessed for example by a pull down assay (e.g. immunoprecipitation assay), optionally pulldown assays in cell culture (immunoprecipitating fora-syn and blotting for CHMP e.g. CHMP2B (or the other way around), in presence and absence of the peptide, or performing fluorescence polarization measurements of the CHMP2B/a-syn interaction in presence and absence of the peptide. The assays can also be compared to a control peptide such as a scrambled sequence peptide.

The peptide may be a naturally occurring sequence or a mutant such as a known mutant or other mutant that maintains the inhibitory activity of the peptide regarding CHMP2B a-syn interaction.

For example, there are known mutations in the CHMP2B MIT-binding motif (MIM), such as Q206H mutant. In addition, various MIM sequence variations were tested.

In an embodiment, the peptide comprises a wildtype MIM sequence.

In an embodiment, the peptide comprises a wildtype a-syn sequence.

In other embodiments, the peptide is a non-naturally occurring peptide for example ones demonstrated herein.

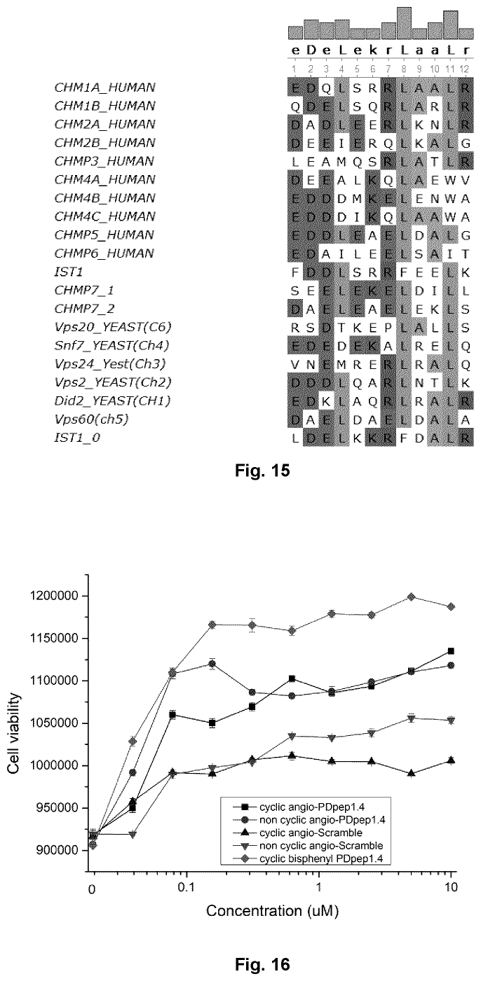

In an embodiment, the CHMP2B: a-syn inhibitor comprises a peptide comprising a MIT-Interacting Motif (MIM) sequence or a sequence with at least 50% sequence identity to said MIM sequence and that inhibits CHMP2B-a-syn interaction by at least 50%. In an embodiment, the peptide has a MIM sequence described in Table 1, FIG. 9 or FIG. 15.

TABLE 1
Exemplary MIM Sequences

| | | | | | Protein |
|---|---|---|---|---|---|
| | | SEQ | | | binding |
| | | ID | Accession | % | domain |
| Name | Sequence | NO: | Number | MIM1 | (PDB) |
| CHM1A_HUMAN | EDQLSRRLAALR | 3 | Q9HD42 | 100 | 2JQ9 |
| | | | | | — |
| | | | | | 2YMB |
| | | | | | — |
| CHM1B_HUMAN | QDELSQRLARLR | 4 | Q7LBR1 | 83 | — |
| | | | | | — |
| | | | | | 3EAB |
| | | | | | 6TZ5 |
| | | | | | 4TXQ |
| | | | | | — |
| | | | | | — |
| CHM2A_HUMAN | DADLEERLKNLR | 5 | O43633 | 100 | — |
| | | | | | — |
| | | | | | — |
| | | | | | — |
| CHM2B_HUMAN | DEEIERQLKALG | 6 | Q9UQN3 | 50 | — |
| | | | | | 2JQK |
| | | | | | — |
| CHMP3_HUMAN | LEAMQSRLATLR | 7 | Q9Y3E7 | 66 | — |
| | | | | | 2XZE |
| CHM4A_HUMAN | QEELAQELLNVG | 8 | Q9BY43 | | |
| CHM4B_HUMAN | QEELDKNLLEIS | 9 | Q9H444 | | |
| CHM4C_HUMAN | QEELNKKMTNIR | 10 | Q96CF2 | | |
| CHMP5_HUMAN | EDDLEAELDALG | 11 | Q9NZZ3 | 66 | 3ULY |
| CHMP6_HUMAN | PVKARPRQAELV | 12 | Q96FZ7 | 33 | — |
| | EDAILEELSAIT | | | 33 | |
| IST1_HUMAN | FDDLSRRFEELK | 13 | P53990 | 66 | 4U7E |
| | | | | | 4U7I |
| | | | | | 4U7Y |
| | | | | | 4WZX |
| | | | | | — |
| CHMP7_HUMAN | SEELEKELDILL | 14 | Q8WUX9 | 50 | |
| | DAELEAELEKLS | | | 66 | |
| Vps20_YEAST | RSDTKEPLALLS | 15 | Q04272 | 33 | — |
| (C6) | | | | | |

In an embodiment, the peptide comprises or comprises up to 1, 2, 3, 4 or 5 mutations (e.g. changes)/10 amino acids relative to a naturally occurring MIM sequence. Peptides, whether or not comprising mutations, that inhibit a-syn and CHMP2B interaction by at least 50%, at least 60%, at least 70% or at least 80% are contemplated.

In an embodiment, the peptide consists of a sequence of 5 to 30, 5 to 25, 5 to 20, 5 to 15, 5 to 10, 7 to 30, 7 to 25, 7 to 20, 7 to 15 or 7 to 10 amino acids. The peptide can for example be 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, or 5 amino acids. In some embodiments, the peptide is at least 7 amino acids. In other embodiments, the peptide is 12 amino acids. In yet other embodiments, the peptide is 9 amino acids

The peptide can be of any length between 5 and for example 30 amino acids.

The peptide can comprise at least 50%, at least 60%, at least 70%, at least 80% or at least 90% sequence identity to said MIM sequence or said a-syn interaction sequence.

Where the peptide comprises additional CHMP or a-syn sequence (e.g. extending N and/or C terminal from said MIM or a-syn interaction sequence), the percent sequence identity can be at least 50%, at least 60%, at least 70%, at least 80% or at least 90% sequence identity to said CHMP or a-syn sequence.

For example, the peptide can be or comprise a C-terminal CHMP sequence comprising a MIM region, optionally a CHMP sequence described herein or one with at least 50%, at least 60%, at least 70%, at least 80% or at least 90% sequence identity to such sequences. Preferably the CHMP sequence is a human CHMP sequence. For example, the peptide can comprise at least 50%, at least 60%, at least 70%, at least 80% or at least 90% sequence identity to a human CHMP C-terminal sequence.

A full length CHMP sequence, optionally CHMP2B sequence can also be used or fragments thereof comprising at least 5, at least 6, at least 7 or more amino acids of the MIM domain. In some embodiments, the CHMP fragment comprises between 5 to 213 amino acids, optionally 199 amino acids, 5 to 30 amino acids, 5 to 25 amino acids, 5 to 20 amino acids, 5 to 15 amino acids, 5 to 10 amino acids, 7 to 30 amino acids, 7 to 25 amino acids, 7 to 20 amino acids, 7 to 15 amino acids, or 12 amino acids. In some embodiments, the CHMP2B fragment is amino acid residues 1-199 in uniprot ID: Q9UQN3.

A-syn sequences up to 60 amino acids can also be used or fragments thereof comprising at least 5, at least 6, at least 7 or more amino acids of the a-syn interaction sequence. In some embodiments, the a-syn interaction sequence fragment comprises between 5 to 60 amino acids, 5 to 50 amino acids, 5 to 40 amino acids 5 to 30 amino acids, 5 to 25 amino acids, 5 to 20 amino acids, 5 to 15 amino acids, 5 to 10 amino acids, 7 to 30 amino acids, 7 to 25 amino acids, 7 to 20 amino acids, 7 to 15 amino acids, or 9 amino acids.

In some embodiments, the length of the peptide is any length between 5 and 213 amino acids long, optionally between 5 and 25 amino acids long, optionally between 5 and 50 amino acids long, optionally between 5 and 75 amino acids long, optionally between 5 and 100 amino acids long, optionally between 5 and 125 amino acids long, optionally, between 5 and 150 amino acids long, optionally between 5 and 200 amino acids long.

In some embodiments, the length of the peptide is any length between 7 and 213 amino acids long, optionally between 7 and 25 amino acids long, optionally between 7 and 50 amino acids long, optionally between 7 and 75 amino acids long, optionally between 7 and 100 amino acids long, optionally between 7 and 125 amino acids long, optionally, between 7 and 150 amino acids long, optionally between 7 and 200 amino acids long.

In an embodiment, wherein the peptide is at least 7 amino acids and/or less than 20 amino acids. In one embodiment, the peptide is 12 amino acids.

In an embodiment, the peptide has a positive overall charge.

In an embodiment, the peptide comprises IPIQLKA (SEQ ID NO: 1), or a sequence with at least 50% sequence identity to IPIQLKA (SEQ ID NO: 1) that inhibits CHMP2B-a-syn interaction, optionally at least 60%, at least 70% or at least 80% or at least 90% sequence identity to IPIQLKA (SEQ ID NO: 1) and that inhibits CHMP2B-a-syn interaction. In some embodiments the peptide comprises IPIQLKA (SEQ ID NO: 1), or a sequence with at least about 50%, optionally about 55%, optionally about 60%, optionally about 65%, optionally about 70%, optionally about 75%, optionally about 80%, optionally about 85%, optionally about 90%, sequence identity to IPIQLKA (SEQ ID NO: 1) and inhibits CHMP2B-a-syn interaction.

In an embodiment, the peptide comprises IERQLKA (SEQ ID NO: 16) (DPpep1.1), EIERQLKALG (SEQ ID NO: 17) (DPpep1.2), DEEIERQLKALG (SEQ ID NO: 6) (DPpep1.3), DEEIERQLDALG (SEQ ID NO: 18) (DPpep1.4), IPKQEKA (SEQ ID NO: 19) (DPpep1.5), EEDDDMKELENWAGSM (SEQ ID NO: 20) (CHMP4-MIM), DEELERRLKALK (SEQ ID NO: 21) (SUPER), EQDELSQRLARLRDQV (SEQ ID NO: 22) (1B-MIM), VPVKARPRQAELVAAS (SEQ ID NO: 23) (6-MIM2), EDQLSRRLAALR (SEQ ID NO: 3) (A1-MIM), DADLEERLKNLR (SEQ ID NO: 5) (2A-MIM), LEAMQSRLATLR (SEQ ID NO: 7) (3-MIM), FDDLSRRFEELK (SEQ ID NO: 13) (IST1-MIM), RNERQLKALG (SEQ ID NO: 24) (optim1), EEEIVRQLKALG (SEQ ID NO: 25) (optim 2), DIEIEFQLKALG (SEQ ID NO: 26) (optim 3), EIERQLKAQI (SEQ ID NO: 27) (optim 4), DEEYERQWKALG (SEQ ID NO: 28) (optim 5), DEAIERVLKALG (SEQ ID NO: 29) (optim 7) DDEIEVQLKALG (SEQ ID NO: 30) (optim.8), TLEIERQLKA (SEQ ID NO: 31) (optim9) or LEEIERQLKALG (SEQ ID NO: 32) (optim10).

In an embodiment, the peptide comprises a sequence with at least 50%, at least 60%, at least 70%, at least 80% or at least 90% sequence identity to IERQLKA (SEQ ID NO: 16) (DPpep1.1), EIERQLKALG (SEQ ID NO: 17) (DPpep1.2), DEEIERQLKALG (SEQ ID NO: 6) (DPpep1.3), DEEIERQLDALG (SEQ ID NO: 18) (DPpep1.4), IPKQEKA (SEQ ID NO: 19) (DPpep1.5), EEDDDMKELENWAGSM (SEQ ID NO: 20) (CHMP4-MIM), DEELERRLKALK (SEQ ID NO: 21) (SUPER), EQDELSQRLARLRDQV (SEQ ID NO: 22) (1B-MIM), VPVKARPRQAELVAAS (SEQ ID NO: 23) (6-MIM2), EDQLSRRLAALR (SEQ ID NO: 3) (A1-MIM), DADLEERLKNLR (SEQ ID NO: 5) (2A-MIM), LEAMQSRLATLR (SEQ ID NO: 7) (3-MIM), FDDLSRRFEELK (SEQ ID NO: 13) (IST1-MIM), RNERQLKALG (SEQ ID NO: 24) (optim1), EEEIVRQLKALG (SEQ ID NO: 25) (optim 2), DIEIEFQLKALG (SEQ ID NO: 26) (optim 3), EIERQLKAQI (SEQ ID NO: 27) (optim 4), DEEYERQWKALG (SEQ ID NO: 28) (optim 5), DEAIERVLKALG (SEQ ID NO: 29) (optim 7) DDEIEVQLKALG (SEQ ID NO: 30) (optim.8), TLEIERQLKA (SEQ ID NO: 31) (optim9) or LEEIERQLKALG (SEQ ID NO: 32) (optim10) and inhibits CHMP2B-a-syn interaction.

In some embodiments, the peptide comprises the amino acid sequence EEIERQLKALG (SEQ ID NO: 2). In some embodiments, the peptide comprises the amino acid sequence KEEEDDDMKELENWAGSM (SEQ ID NO: 33). In an embodiment, the peptide comprises a sequence with at least 50%, at least 60%, at least 70%, at least 80% or at least 90% sequence identity to EEIERQLKALG (SEQ ID NO: 2) or KEEEDDDMKELENWAGSM (SEQ ID NO: 33).

In an embodiment, the peptide comprises or is DEEIERQLKALG (SEQ ID NO: 6) (DPpep1.3).

In an embodiment, the peptide is or comprises amino acid residues 103-114 of a-syn (Accession number NM_000345.4) or a fragment thereof that is at least 5 amino acids long. In some embodiments, the peptide is or comprises amino acid residues 103-108, 103-109, 104-110, 104-113, 105-111, 105-112, 106-111, 106-112, 107-111, 107-113, 108-113 or 108-114 of a-syn (Accession number NM_000345.4). In some embodiments, the peptide has the amino acid sequence In an embodiment, the peptide is at least 5 amino acids of NEEGAPQEGILE (SEQ ID NO: 34), In an embodiment, the peptide is or comprises NEEGAPQEGILE (SEQ ID NO: 34) (corresponding to positions 103 to 114).

The peptide can comprise for example a sequence with at least about 50%, about 55%, about 60%, about 65%, about 70%, about 75%, about 80%, about 85%, about 90%, about 95%, about 98% or greater identity to a MIM, CHMP, a-syn interaction sequence. Peptides that inhibit for example CHMP2B:a-syn interaction by at least 50% or greater as described herein are contemplated.

The peptides described herein may have 1, 2, 3 or 4 or more amino acid substitutions, optionally conservative substitutions. For example, a peptide with at least 50% sequence identity to a sequence of 7 amino acids in length, may have 1, 2, 3 or 4 substitutions, optionally conservative substitutions. The peptide may comprise for example up to 5 substitutions for every 10 amino acids. This can be calculated by looking at the full length of the peptide such that some stretches may have more than 5 substitutions/10 amino acids and others that are less than 5, with the overall number of substitutions not exceeding 5/10 amino acids.

Substitutions and mutations are used interchangeable.

The peptide may comprise one or more modifications, for example 2, 3, 4, 5, 6 or more modifications compared to a sequence described herein. In some embodiments, each amino acid of the peptide is modified. In some embodiments, the modification is stapling the peptide (for example, as shown for the cyclic bisphenyl conjugated peptides).

For example, the modification can be an amino acid modification, or a stability modification or both. As demonstrated in the results, the peptides can be modified to increase stability by stapling the peptides. Stapling the peptide can be achieved by covalently linking the sidechains of two amino acids using for example, a hydrocarbon moiety, a bishphenyl moiety, or an hexafluorobenzene, thereby forming a peptide macrocycle.

“Stapling” of a peptide refers to covalently linking two residues in a helical peptide (located such that they are on the same side of the helix, e.g., positions i (where i denotes the position of the first residue) and i+3 or positions i and i+7, using a linking moiety. This linking moiety can be a hydrocarbon moiety (hydrocarbon staple), or a different chemistry, such as a bisphenyl moiety. The modification can be carried out after the synthesis of the peptide. The stapling positions can for example be cysteine residues that are present or added to the peptide. After synthesis, the peptide can be chemically conjugated to the linking moiety, for example in the case of two cysteines being linked, the two cysteines are linked to the linking moiety (60).

In some embodiments the peptide has a positive charge. In some embodiments, the peptide lacks a C-terminal positive charge.

In an embodiment, the peptide is a stapled peptide. In some embodiments, the modification comprises stapling using for example a hydrocarbon or other stapling moiety.

Any of the peptides described herein can be stapled. In an embodiment, wherein the stapled peptide comprises a sequence selected from DEEYCRQWKALC (SEQ ID NO: 35), DEEICRQLDALC (SEQ ID NO: 37) or DIEICFQLKALC (SEQ ID NO: 36).

In some embodiments, the modification comprises inclusion of one or more non-canonical or D-amino acids. For example, the modification can comprise substituting one or more amino acids of the any of the peptides described herein with a D-amino acid or a non-canonical amin acid.

In some embodiments, the peptides are modified to increase stability, optionally wherein the modification comprises inclusion of non-canonical amino acids or generation of D-amino acids.

In some embodiments, the inhibitor, peptide or polypeptide is or comprises a peptide that reduces a-syn toxicity and/or CHMP self-interaction and/or CHMP2B/a-syn interaction. In some embodiments, the peptide inhibitor is any peptide that's reduces CHMP2B/a-syn interaction. In some embodiments, the inhibitor, peptide or polypeptide is or comprises any peptide that reduces a-syn toxicity and/or CHMP self-interaction and/or CHMP2B/a-syn interaction by at least about 50%, optionally about 55%, optionally about 60%, optionally about 65%, optionally about 70%, optionally about 75%, optionally about 80%, optionally about 85%, optionally about 90%, optionally about 95%, optionally about 96%, optionally about 97%, optionally about 98% or optionally about 99%.

As described herein the said peptide can comprise one or more exogenous residues, optionally interspersed within said peptide, for example for one or more, optionally two cysteine residues interspersed within said peptide, optionally wherein at least 4 amino acids (e.g. MIM sequence or a-syn sequence) is present between the exogenous residues. Exogenous residues are added for example for cyclizing a peptide. They may be interspersed within or added to an end or ends of a peptide. The peptide with or without exogenous residues is one that inhibits CHMP2B-a-syn interaction by at least 50% as described herein.

The inhibitor may comprise and/or the peptide may be conjugated to a transport moiety (optionally an amino acid sequence), for example a cell-penetrating moiety (such as tat peptide, cyclic cell penetrating peptide) and/or a blood-brain barrier penetrating moiety (such as an Angiopep peptide (Angiochemem; Montreal Candada) or blood brain crossing antibody such as an antibody or nanobody, optionally a transferrin antibody). Examples of engineered antibodies that can cross the blood brain barrier have been made for example as described in Ledford, H. Engineered antibodies cross blood-brain barrier. Nature (2011) herein incorporated by reference. Examples of cell penetrating peptides can be found in for example Xie, Jing et al. “Cell-Penetrating Peptides in Diagnosis and Treatment of Human Diseases: From Preclinical Research to Clinical Application” Frontiers in pharmacology vol. 11 697. 20 May. 2020, which is hereby incorporated by reference. Examples of cyclic cell penetrating peptides can found in for example Qian, Ziqing et al. “Discovery and Mechanism of Highly Efficient Cyclic Cell-Penetrating Peptides.” Biochemistry vol. 55,18 (2016): 2601-12, which is incorporated herein by reference. A review of strategies to deliver peptide drugs to the brain can be found at Laltsa, A et al Mol. Pharmaceutics 2014, 11, 4, 1081-1093, which is incorporated herein by reference.

In some embodiments, the peptide is conjugated to an aprotinin sequence or fragment thereof such as an Angiopep peptide. In one embodiment, the angiopep and the conjugated peptide has one of the following sequences: TFFYGGSRGKRNNFKTEEYDEEICRQLDALC (SEQ ID NO: 38) or TFFYGGSRGKRNNFKTEEYGEARCEIQDLLC (SEQ ID NO: 39) and is cyclic. In another embodiment, the angiopep and the conjugated peptide has one of the following sequences: TFFYGGSRGKRNNFKTEEYDEEIERQLDALG (SEQ ID NO: 40) or TFFYGGSRGKRNNFKTEEYGEARDEIQDLLE (SEQ ID NO: 41) and is non-cyclic. The peptide can be labelled, for example with FITC, for example for tracking.

Peptides can be synthesized or purchased from a custom peptide synthesis service available for example LifeTein (New Jersey). As described herein, the cyclic peptides were synthesized using an aminohexanoic acid spacer and for cyclic peptides creating a disulphide bridge between cysteine residues e.g. cyclic peptides can be synthesized as follows (N-Terminal: FITC-Ahx, C-Terminal: Amidation, Disulfide Bridge between Cysteine residues) and non-cyclic peptides can be synthesized as follows (N-Terminal: FITC-Ahx, C-Terminal: Amidation).

The cyclic peptides can be used as linear peptides. Alanine scan studies showed that modifications of these residues did not affect the activity of the peptides. Accordingly, the C residues for example used for cyclization can be replaced with other residues, for example alanine.

The inhibitor may also comprise and/or the peptide may be conjugated to other attachments. In some embodiments, the inhibitor comprises and/or the peptide is conjugated to a serum half-life extending moiety such as a lipid, Fc portion of an antibody or PEG by PEGylation.

The inhibitor may also comprise or consist of a polypeptide comprising the peptide and for example a cell penetrating peptide, a blood brain barrier penetrating peptide optionally a nanobody, or other type of peptide or antibody. The methods and uses disclosed herein can also comprise administering or use of the inhibitors, peptides and polypeptides described herein.

A further aspect is a polypeptide comprising a peptide described herein. For example, in an embodiment, the peptide consists of a 5 to 213 amino acids, preferably 5 to 30 amino acids. The peptide can comprise other lengths as described elsewhere.

Said peptide can comprise or consist of a MIT-Interacting Motif (MIM) sequence or a sequence with at least 50% sequence identity to said MIM sequence that inhibits CHMP2B-a-syn interaction by at least 50% as described herein. Said peptide can comprise or consist of a a-syn interaction sequence or a sequence with at least 50% sequence identity to said a-syn interaction sequence.

As also described herein the said peptide can comprise one or more exogenous residues, optionally interspersed for example for one or more, optionally two cysteines. Exogenous residues are added for example for cyclizing a peptide. They may be interspersed within or added to an end or ends of a peptide. The peptide with or without exogenous residues is one that inhibits CHMP2B-a-syn interaction by at least 50% as described herein.

The polypeptide can comprise any peptide described herein. In some embodiments, the polypeptide can comprise a TAT peptide or other cell-penetrating peptide, and/or blood brain barrier penetrating peptides such as angiopep, and/or a blood brain barrier crossing moiety such as a nanobody.

Similar to peptides described herein, the polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Mode1396; Milligen/Biosearch 9600). Alternatively, the peptides, polypeptides, fragments or variants thereof described herein may be recombinantly produced using various expression systems as is well known in the art.

Also provided in another aspect is a charged multivesicular body protein 2B: a-synuclein (CHMP2B:a-syn) inhibitor comprising a peptide described herein.

The peptide can be any peptide described herein.

In some embodiments, the polypeptide or inhibitor comprises and/or the peptide is fused to a cell penetrating or blood-brain barrier penetrating moiety or to a serum half-life extending moiety such as a lipid, Fc portion of an antibody or PEG by PEGylation.

Another aspect is a nucleic acid molecule comprising a polynucleotide sequence encoding a polypeptide or peptide described herein. In an embodiment, the polynucleotide is codon optimized, for example for humans. The nucleic acid molecule can be used in methods described herein.

In some embodiments, the polynucleotide is delivered via any viral vector, optionally an AAV vector.

Also provided in another aspect is a vector comprising a vector backbone and a nucleic acid molecule described herein. For example, the vector backbone can be a lentivirus or an adeno associated virus.

The inhibitor can be a nucleic acid molecule or vector described herein.

A further aspect is a recombinant cell recombinantly expressing the polypeptide or peptide or comprising the nucleic acid molecule or vector described herein.

The cell targeted in the methods or the cells used to make the recombinant cell can be any cell. In some embodiments, the cells are neurons e.g. neurons can be targeted or the recombinant cell is a recombinant neural cell. In other embodiments, the cells are astrocytes, microglial cells, ependymal cells, or oligodendrocytes. In other embodiments, the cells are satellite cells or Schwann cells.

Recombinant cells can be made by for example being transformed, transfected or transduced with a vector comprising a nucleic acid, optionally any nucleic acid described herein. In some embodiments, the recombinant cells are HEK293T, HEK293S, HEK293F and/or CHO cells and may be used to produce recombinant peptides.

A further aspect is a composition comprising a peptide, a polypeptide, a CHMP2B:a-syn inhibitor, a nucleic acid molecule, a vector or a recombinant cell described herein.

The composition can also comprise a suitable diluent or carrier. In some embodiments, the carrier is a pharmaceutically acceptable carrier.

Also provided are uses of the inhibitors, peptides, polypeptides, nucleic acid molecules, vectors, recombinant cells and compositions described herein as well as their use for manufacturing a medicament, for example for treating a synucleinopathy.

In an embodiment, the a-syn that is decreased is oligomerized a-syn. For example, the a-syn decreased may be oligomeric a-syn or the toxicity reduced may be due to a-syn. The a-syn can also be non-oligomerized a-syn.

The methods can be used where the cell is in vivo. For example the cell can be contacted by administering the by administering the CHMP2B:a-syn inhibitor to a subject in need thereof. The inhibitor is for a peptide inhibitor that inhibits interaction between CHMP2B and a-syn, for example decreasing the interaction in its presence by at least 50%.

The subject in need thereof can be a subject with a synucleinopathy.

For example, the synucleinopathy can be Parkinson's disease (PD). In some embodiments, the synucleinopathy is multiple system atrophy (MSA), dementia with Lewy bodies (DLB), the Lewy body variant of Alzheimer's Disease (AD), neurodegeneration with brain iron accumulation, Parkinson's disease dementia (PDD), Alzheimer's disease and/or prodromal PD/DLB/MSA (e.g., REM sleep behaviour disorder, primary autonomic failure, MCI-LB or DLB-MCI). In addition, there are biomarkers in development to identify people with alpha-synuclein aggregation ante-mortem (e.g., RT-QuIC assay using CSF and possibly PET imaging eventually). As such, in some embodiments, patients identified as having a-synuclein aggregation could benefit from therapies that reduce alpha-synuclein accumulation/aggregation such as inhibitors, peptides, polypeptides, nucleic acid molecules, vectors, recombinant cells and compositions described herein. There will also be the possibility of utility in prodromal conditions including but not limited to REM sleep behaviour disorder (RBD), primary autonomic failure and Gaucher disease.

The subject can comprise a disease causing mutation in a-syn gene. Mutations in a-syn gene have been associated with synucleinopathies. For example, the mutation can be one or more of the following: A30G, A30P, E46K, H50Q, G51D, A53E, A53T, A53V and/or SNCA multiplication (duplication, triplication) and/or a truncation mutation.

Also provided in an aspect is a method of inhibiting neural degeneration, the method comprising administering to a subject in need thereof a charged multivesicular body protein 2B: a-synuclein (CHMP2B:a-syn) inhibitor as described herein.

As demonstrated herein the inhibitor can decrease oligomerized a-syn and prevent neural degeneration.

In an embodiment, the subject in need thereof is a subject with a synucleinopathy such as Parkinson's disease (PD), multiple system atrophy (MSA), dementia with Lewy bodies (DLB), the Lewy body variant of Alzheimer's Disease (AD), neurodegeneration with brain iron accumulation, Parkinson's disease dementia (PDD), Alzheimer's disease and/or prodromal PD/DLB/MSA (e.g., REM sleep behaviour disorder, primary autonomic failure, MCI-LB or DLB-MCI).

The inhibitors may be administered for example via IV administration, directly to the brain for example either transiently or with a permanent infusion catheter and pump, optionally the parenchyma and/or ventricle of the brain. In some embodiments, the inhibitor may be administered using gene therapy using for example nucleic acids and vectors described herein. In other embodiments, the inhibitor may be delivered via augmented delivery, optionally using focused ultrasound. In some embodiments, the inhibitor comprises a cell and blood-brain-barrier crossing moiety and is optionally administered intravenously. In some embodiments, the inhibitor is a stapled peptide which cross the cell membrane and may be injected into the brain directly or delivered using focused ultrasound. In some embodiments, inhibitor can be delivered using gene therapy vectors such as AAVs.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

Proteomic Screen Identifies Peptides that Rescue a-Syn Cytotoxicity

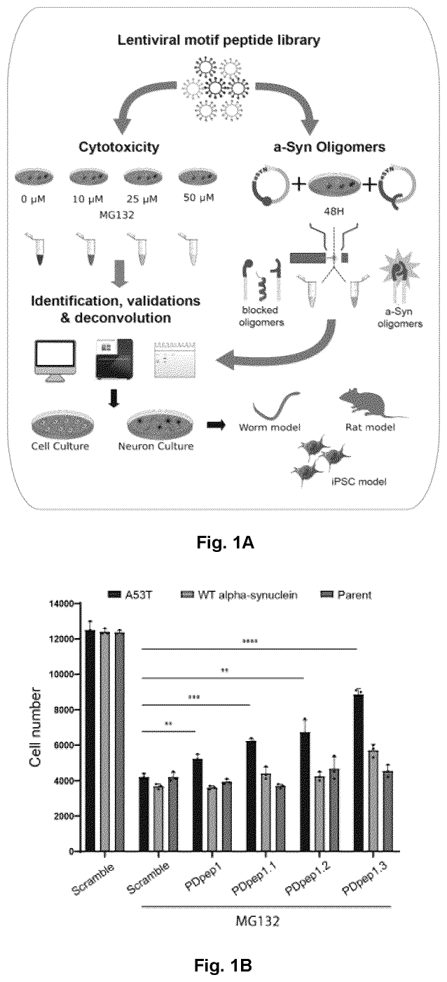

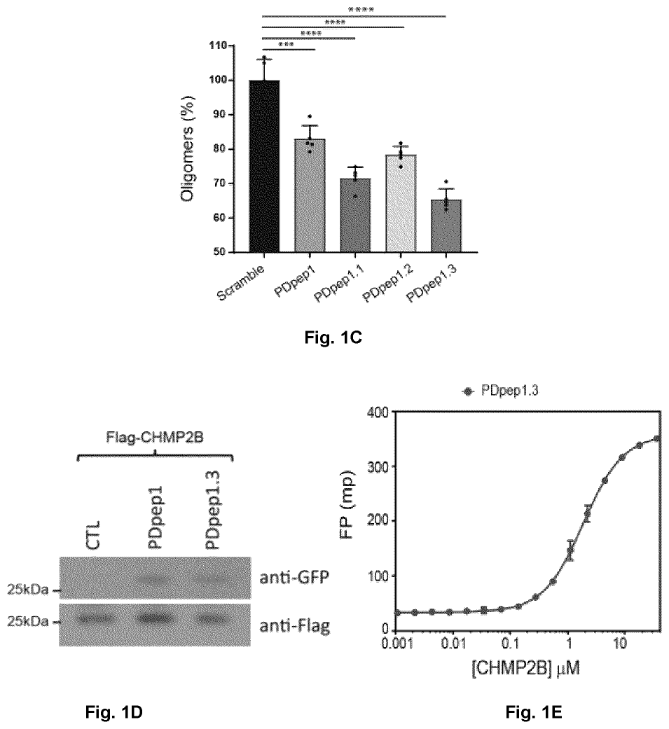

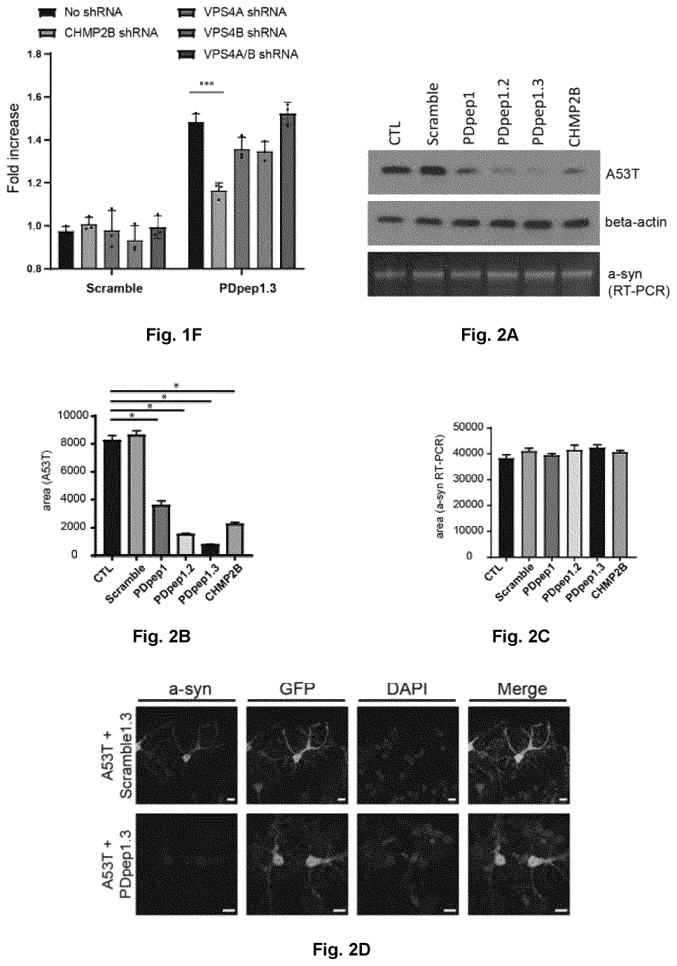

A screening system to discover candidate peptides to perturb endogenous PPIs based on libraries of short linear interaction motifs that mediate a large fraction (˜30%) of human PPIs (10-12) was recently developed. Here, a lentiviral library of 50,549 7-mer peptide motifs (13) on a green fluorescent protein (GFP) scaffold (FIG. 1A) was used. Cells in a pooled format were infected and the amino acid sequence of each peptide served as its own barcode. By using a low multiplicity of infection followed by puromycin selection, each infected cell expressed one unique GFP-peptide. Peptides that reduced a-syn-mediated cytotoxicity or a-syn oligomers in parallel (FIG. 1A), were screened for.

To cause cytotoxicity relevant to PD, proteostatic stress in HEK293 cells was induced by inhibiting the proteasome with MG132 and inducing the overexpression of human a-syn (WT) from idiopathic PD in one screen and the more toxic A53T mutant from familial PD in a second screen. The combination of a-syn overexpression and pharmacological proteasome inhibition led to death of most cells, with only those that expressed a protective peptide surviving. By extracting genomic DNA from the surviving cells and amplifying the peptide coding sequences, the protective peptides (FIG. 1A) were identified.

To detect a-syn oligomers, a protein fragment complementation assay (PCA) of a-syn oligomerization in HEK293 cells (14) was used. In this assay, human A53T a-syn was fused to either the C- or N-terminal half of yellow fluorescent protein (YFP). No fluorescence is detectable while a-syn exists in monomers but, when a-syn oligomerizes, the two halves of YFP come in close enough proximity to form a functional, spontaneously fluorescent protein. This fluorescence has previously been shown to approximate levels of toxic oligomeric conformations of a-syn in cell and rodent models (15-17). By using fluorescence activated cell sorting (FACS), cells expressing Flag-tagged peptides that directly or indirectly interfered with a-syn oligomer formation were isolated and then the peptides were identified from genomic DNA (FIG. 1A). The screens yielded 10 hits (Table 1). The hit with the most reads (i.e., the most abundant) was the same peptide in all screens: IPIQLKA (SEQ ID NO: 1) (PDpep1) (Table 2). The cytoprotective effects of Pdpep1 in HEK293 cells overexpressing WT or A53T a-syn (FIG. 1B, 7A) were validated, as well as its ability to reduce WT a-syn oligomers in a luciferase-based PCA (18, 19) (FIG. 1C). Lead peptide targets CHMP2B, a member of ESCRT-III

The amino acid sequence of Pdpep1 was mapped to the C-terminal region of Charged Multivesicular Body Protein 2B (CHMP2B), a member of the Endosomal Sorting Complex Required for Transport-III (ESCRT-III) machinery. This region corresponds to a MIT-Interacting Motif (MIM) (20) that is thought to mediate several protein-protein interactions. Using this mapping, optimization of Pdpep1 by generating several versions of the peptide of various lengths based on the relevant sequence of CHMP2B: IERQLKA (SEQ ID NO: 16) (Pdpep1.1), EIERQLKALG (SEQ ID NO: 17) (Pdpep1.2), and DEEIERQLKALG (SEQ ID NO: 6) (Pdpep1.3) was sought. It was found that Pdpep1.3 had the most prominent effects on cell survival under proteostatic stress (mediated by MG132) in cells expressing A53T a-syn and on a-syn oligomers (FIG. 1B, 1C).

ESCRT members have been implicated in neurodegenerative proteinopathies, such as Hrs of ESCRT-0 and Tsg101 of ESCRT-I in models of Alzheimer's disease (21). ESCRT controls formation of multivesicular bodies (MVBs), a subset of late endosomes that contain cargo-laden intralumenal vesicles (ILVs). MVBs can fuse with lysosomes leading to degradation of ILVs and their protein cargoes. In addition, ESCRT-III is important for lysosome maintenance and repair of lysosomal membranes (22). Mutations in the CHMP2B gene are rare but established causes of amyotrophic lateral sclerosis (ALS) and frontotemporal dementia (FTD) (23, 24). A genome-wide association study has also nominated a CHMP2B variant as a genetic risk factor for PD (25). The CHMP2B protein is expressed in all neurons, and brains of mice expressing mutant CHMP2B form neuronal inclusions due to impaired lysosomal degradation (26). Furthermore, CHMP2B has been found to co-localize with a-syn in Lewy bodies in brains of PD patients (27).

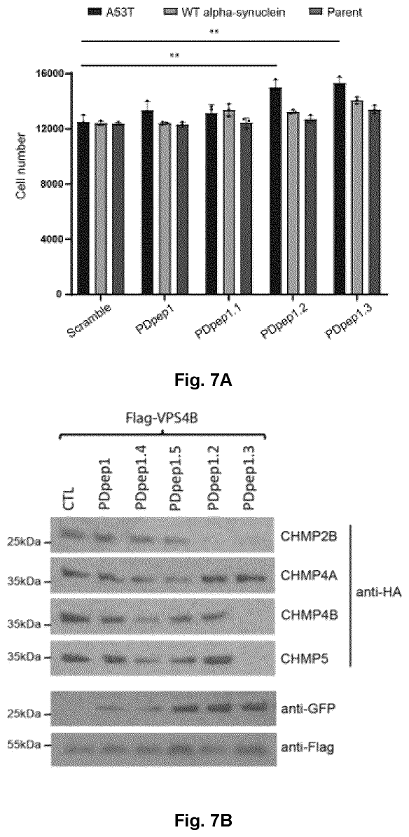

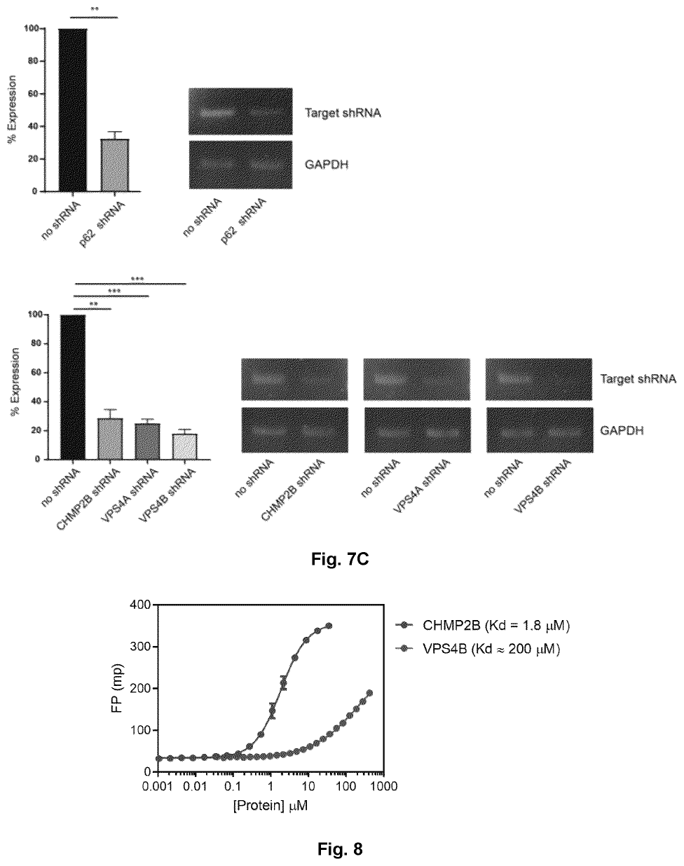

CHMP2B has been reported to bind to Vacuolar Protein Sorting 4 (VPS4), an AAA-type adenosine triphosphatase (20). This interaction is mediated by the MIM on CHMP2B and a Microtubule Interacting and Trafficking (MIT) domain on VPS4. It was found that Pdpep1.3 inhibits the CHMP2B-VPS4 interaction (FIG. 7B). However, it was also noted that many CHMP proteins can self-interact and have been reported to multimerize, which may contribute to their function (28). In particular, CHMP2B can self-interact (28) and is thought to polymerize at the budding of membranes during endosome formation (29). It was found that Pdpep1.3 binds to CHMP2B both in cells (FIG. 1D) and in vitro (FIG. 1E). To test whether the effect of Pdpep1.3 on cell survival was mediated through CHMP2B or VPS4, shRNA knockdown of each was performed. It was found that the cytoprotective effect of Pdpep1.3 was prevented by CHMP2B knockdown, but not by knockdown of VPS4A and/or VPS4B (FIG. 1F, 7C). It was noted that the CHMP2B-VPS4 interaction, though proven to be functional, is quite weak (178 μM) even for a motif-domain interaction (20), and the affinity measurements revealed that Pdpep1.3 binds to CHMP2B much more tightly (1.8 μM) than it does to VPS4 (>100 μM) (FIG. 8). Taken together, these results imply that Pdpep1.3 protects against a-syn cytotoxicity by targeting interactions of CHMP2B. Consistent with the poor affinity to VPS4, the sequence of Pdpep1.3 differs from classic MIMs in that it lacks a C-terminal positive charge (FIG. 9A), making it a weak binder of MIT domains. Other MIM peptides were also tested, all of which showed markedly weaker effects than Pdpep1.3 in rescuing a-syn cytotoxicity (FIG. 9B), further suggesting that it is not a MIM-MIT domain interaction that is targeted. It was noted that the CHMP2B self-interaction, mediated by the Pdpep1.3 sequence, is much stronger than its interaction with VPS4 and thus the C-terminus of CHMP2B may play a role in its polymerization.

Pdpep1.3 Promotes Degradation of a-Syn

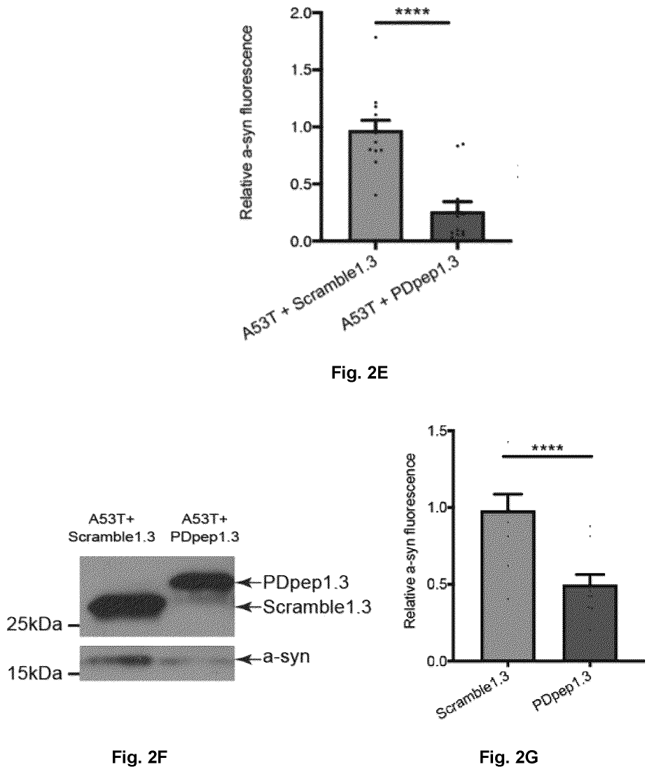

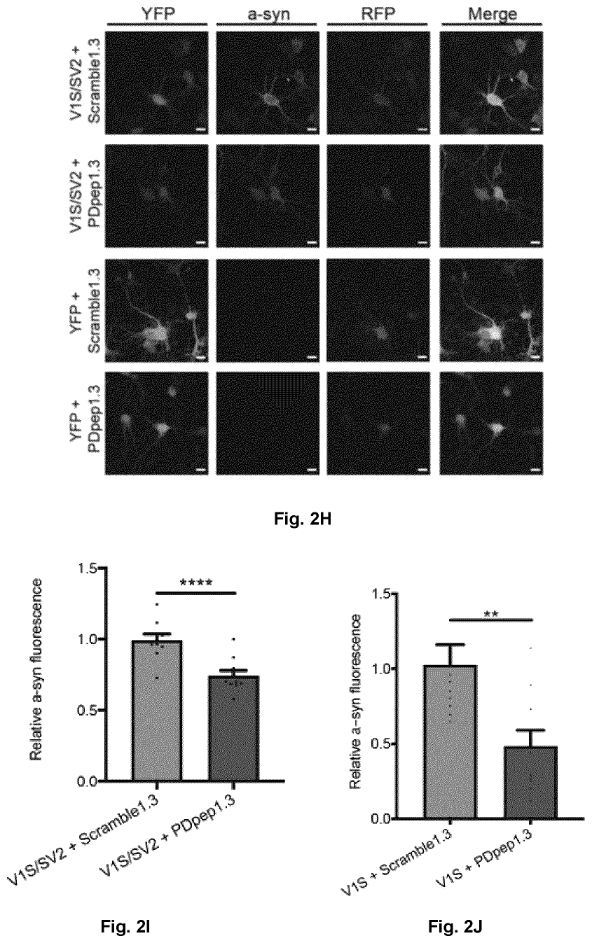

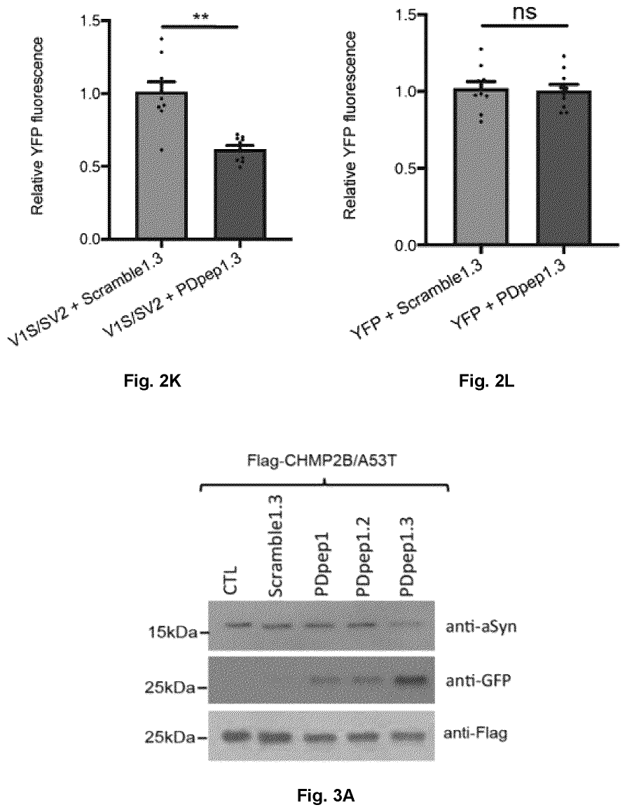

Determination of the downstream effects of Pdpep1 were sought. Since ESCRT is involved in protein degradation, playing an important role in the endolysosomal pathway, it was hypothesized that Pdpep1.3 might promote degradation of a-syn. To test this, A53T a-syn protein levels in HEK293 cells co-transfected with Pdpep1 and its optimized versions was examined. Cells transfected with each of the peptides or with full-length CHMP2B demonstrated reduced a-syn protein levels by immunoblotting, while the control peptide, Scramble1.3 (same amino acid composition but scrambled sequence), had no effect (FIG. 2A, 2B). Levels of a-syn mRNA, measured by RT-PCR, were not different across conditions and therefore this reduction was not due to decreased transcription (FIG. 2A, 2C). To ensure the effect of Pdpep1.3 was not limited to cell lines, primary cortical neurons isolated from E17 rat embryos with adeno-associated viruses (AAVs) encoding human A53T a-syn and either GFP-tagged Pdpep1.3 or Scramble1.3 control were co-transduced. Immunostaining of these neurons showed a significant decrease in A53T a-syn fluorescence intensity in those neurons expressing Pdpep1.3 compared with Scramble1.3 (FIG. 2D, 2E). A corresponding reduction in total A53T a-syn protein levels with Pdpep1.3 was seen by immunoblotting of the neuronal lysates (FIG. 2F). Additionally, these neurons for endogenous a-syn and found reduced fluorescence intensity with Pdpep1.3 (FIG. 2G) were immunostained. It was also examined whether Pdpep1.3 could reduce WT a-syn levels in primary neurons and simultaneously measured levels of a-syn oligomers and of total a-syn. To this end, neurons with AAVs encoding human WT a-syn fused to the N- or C-terminal half of YFP (V1S or SV2, respectively) were transduced. A significant lowering in total a-syn fluorescence was observed with co-expression of V1S and SV2 (FIG. 2H, 2I) or expression of V1S alone (FIG. 2J) with red fluorescent protein (RFP)-tagged Pdpep1.3 compared with Scramble1.3. In addition, Pdpep1.3 reduced YFP fluorescence from co-expressed V1S and SV2 but had no effect on fluorescence of YFP alone (FIG. 2H, 2K, 2L). Thus, total levels of WT and mutant a-syn protein, including endogenous a-syn and not limited to a-syn oligomers, in cultured neurons are decreased by Pdpep1.3.

Pdpep1.3 Disrupts a Newly Identified Interaction Between a-Syn and CHMP2B

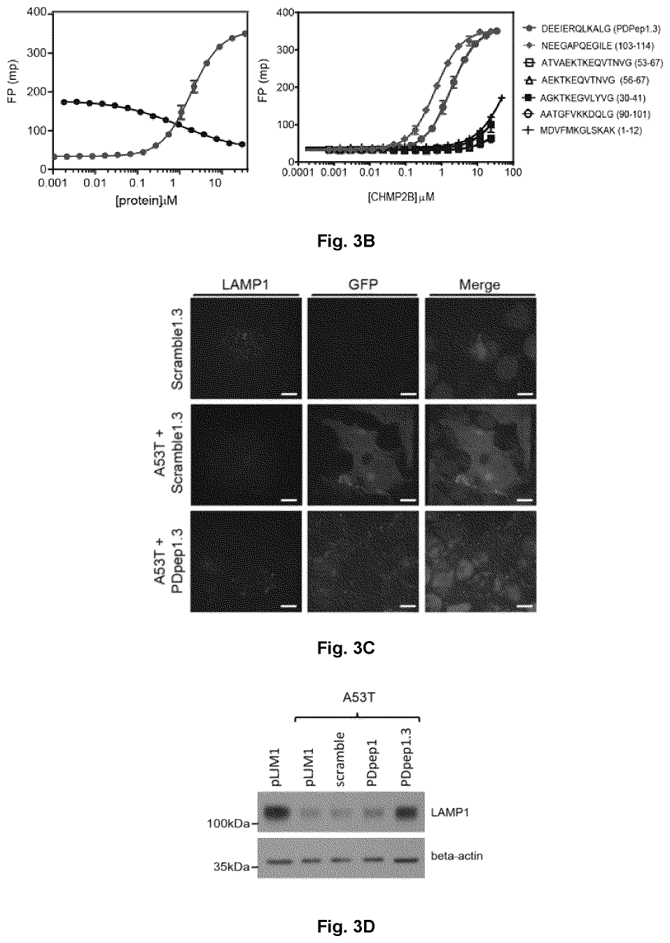

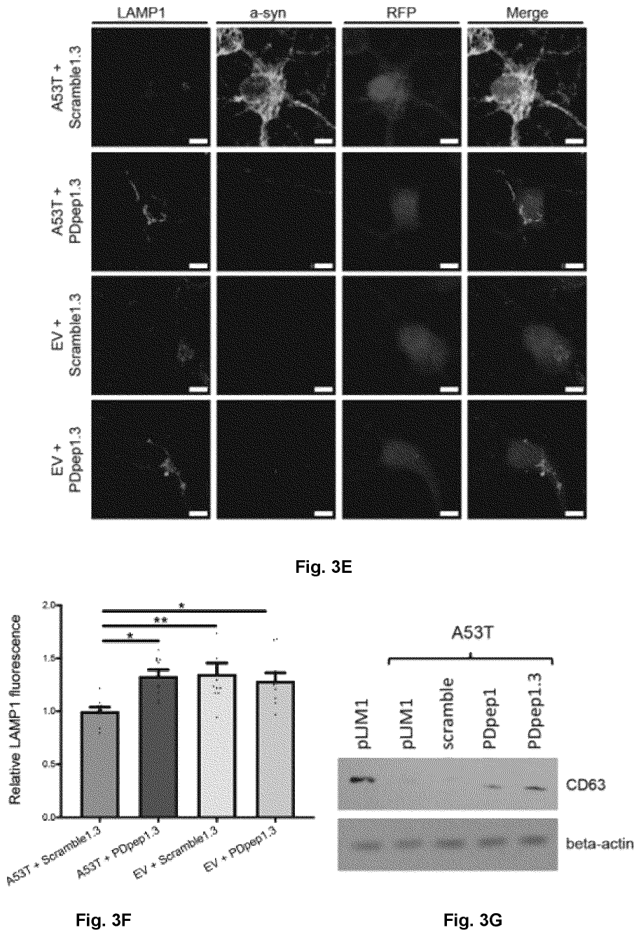

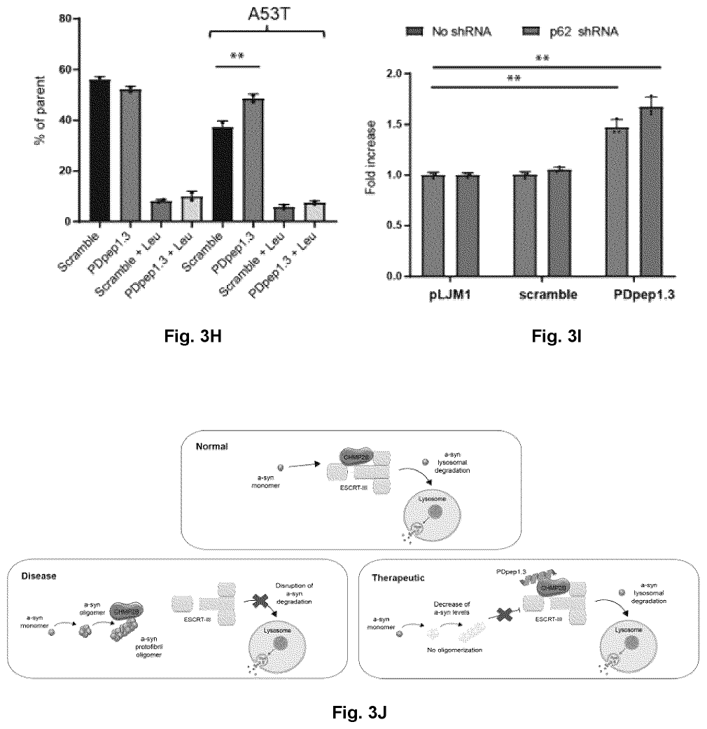

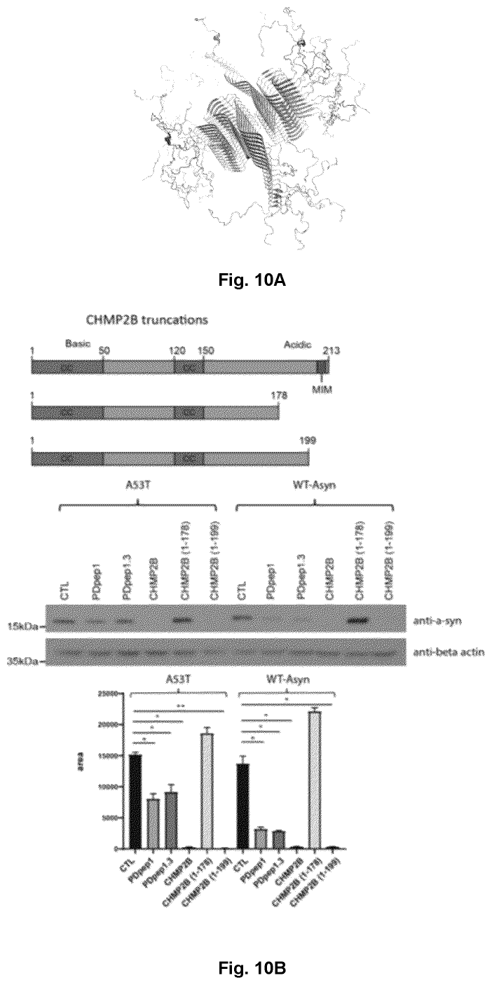

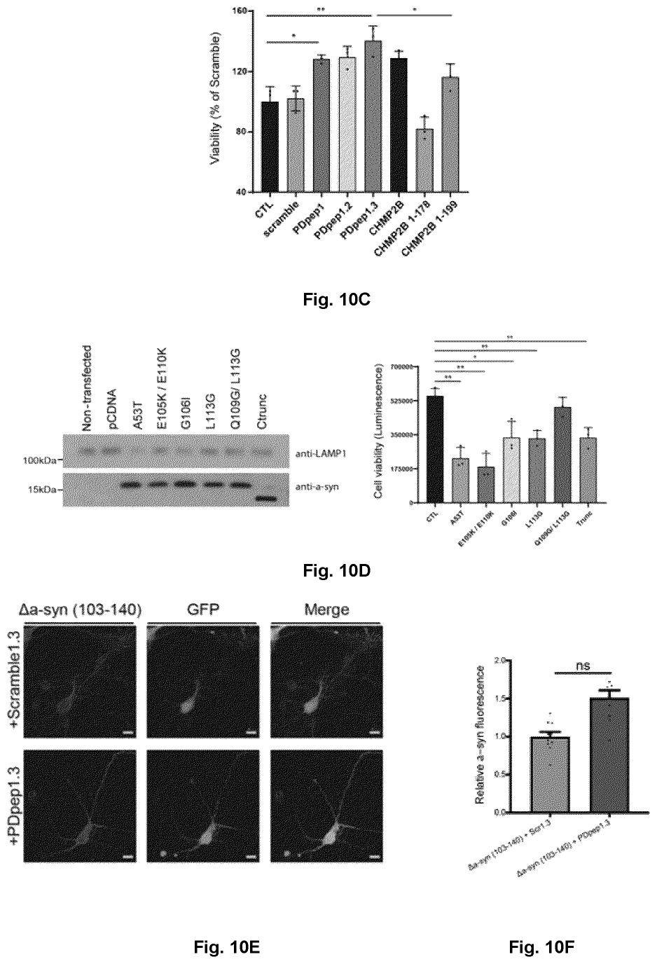

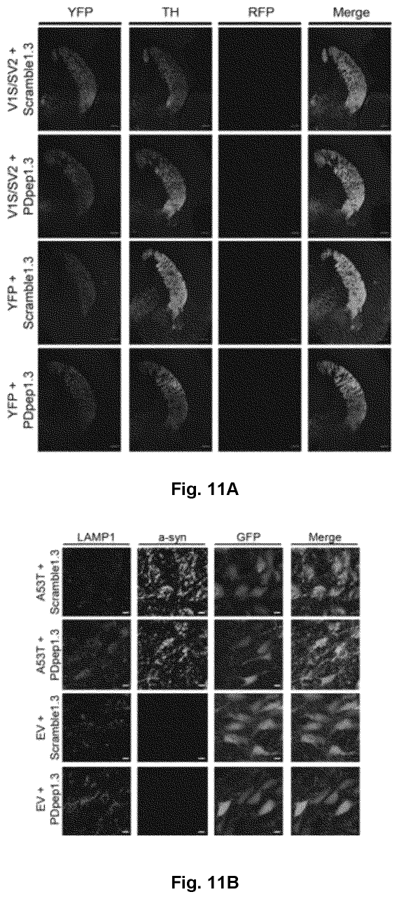

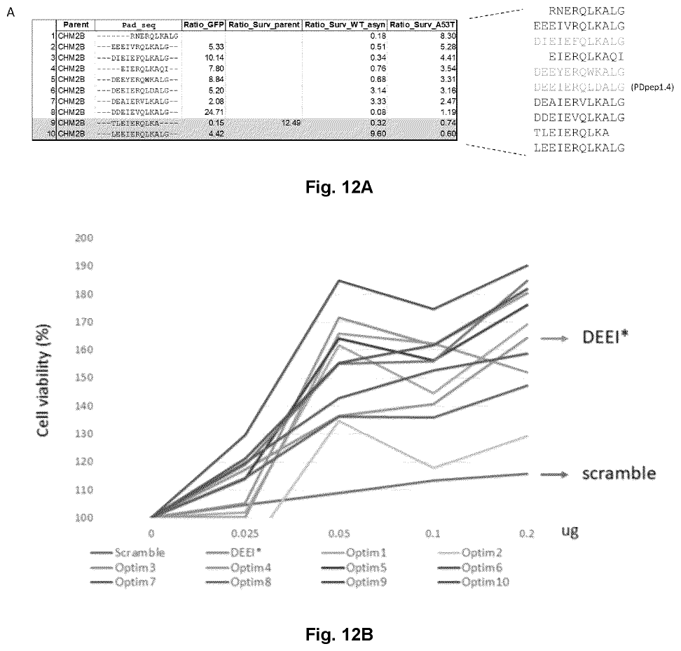

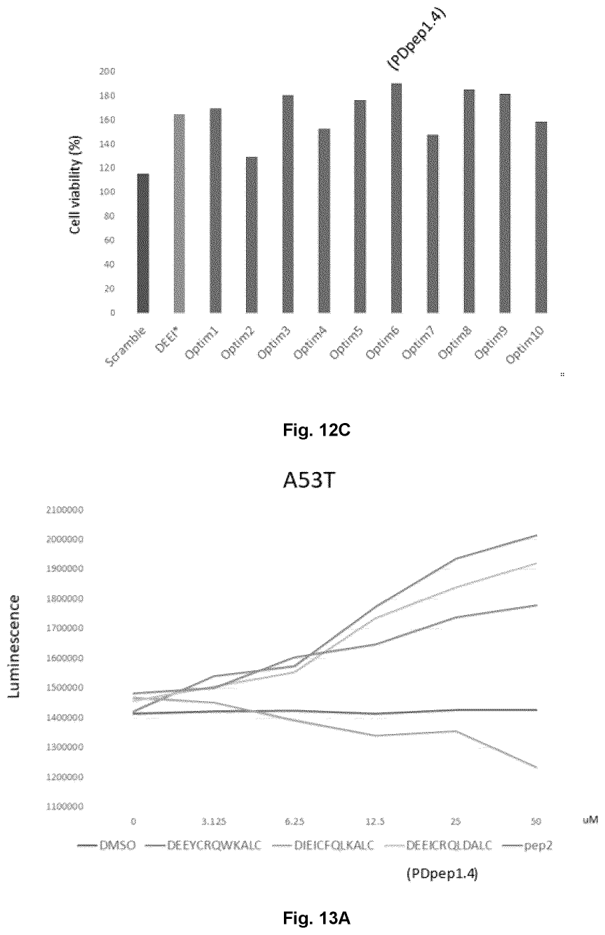

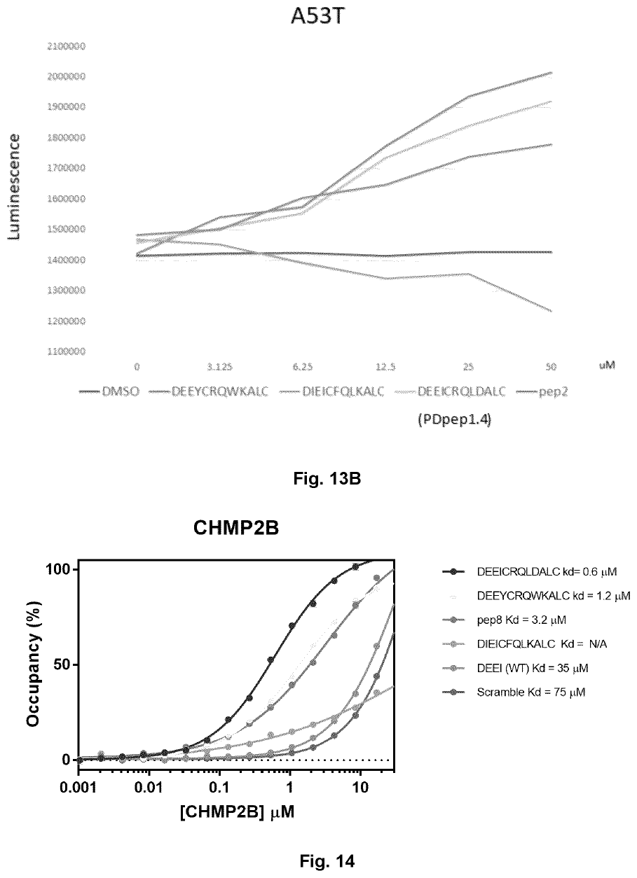

Increased levels of a-syn have been reported to disrupt ESCRT and the endolysosomal pathway (30), but the exact mechanism of this disruption remains unknown. It was hypothesized that it could be mediated by a direct interaction between a-syn and CHMP2B; it has been suggested previously that a-syn may exert an effect on the endolysosomal pathway through CHMP2B (30). Co-immunoprecipitation experiments were first performed and it was found that a-syn interacts directly with CHMP2B (FIG. 3A). By testing several a-syn peptide fragments, the interaction site was mapped to amino acids 103-114 of a-syn, which bound to CHMP2B with an affinity of 0.64 μM (FIG. 3B). This binding region of a-syn is within its C-terminal region and is exposed in structures of a-syn oligomers (FIG. 10A) (31), suggesting that such oligomers bind to CHMP2B more tightly than monomers due to avidity. Thus, higher levels of a-syn along with increased oligomerization could trigger this interaction and sequester CHMP2B, thereby leading to a disruption of ESCRT-III. Consistent with this, it was found that addition of full-length CHMP2B reduces a-syn levels (FIG. 10B) and rescues a-syn cytotoxicity (FIG. 10C). Addition of truncated CHMP2B (removing the MIM (1-199)) also rescues toxicity, while a shorter truncation (1-178), which has been implicated in FTD (23), actually increased toxicity (FIG. 10C) perhaps through a gain of function mechanism where the protein is now actively toxic.

Whether Pdpep1.3 could perturb the a-syn-CHMP2B interaction and thereby restore CHMP2B function in the ESCRT pathway was questioned. Pdpep1.3 directly interacted with CHMP2B in vitro with an affinity similar to the a-syn 103-114 peptide (FIG. 3B), and Pdpep1.3 and a-syn indeed competed for binding to CHMP2B (FIG. 3B). Moreover, it was found that Pdpep1 and its optimized versions disrupted the a-syn-CHMP2B interaction in HEK293 cells; Pdpep1.3 caused the most prominent disruption (FIG. 3A).

Pdpep1.3 Rescues Lysosomal Activity Disrupted by a-Syn

Since both ESCRT and a-syn can influence lysosomal function, assessment of the effect of Pdpep1.3 on lysosomes was sought. The levels of the lysosomal marker, Lysosomal-Associated Membrane Protein 1 (LAMP1) were first measured. Decreased LAMP1 is associated with accumulation of a-syn in brains of PD patients and rodent models (32). A similar decrease in LAMP1 with overexpression of A53T a-syn in HEK293 cells (FIG. 3C, 3D) was found. LAMP1 levels were restored upon co-expression of Pdpep1.3. Furthermore, rat cortical neurons expressing human A53T a-syn also demonstrated decreased levels of LAMP1 (FIG. 3E, 3F). Pdpep1.3, but not Scramble1.3, reduces a-syn levels (FIG. 2D, 2E) and this was associated with restored LAMP1 levels (FIG. 3E, 3F), suggesting rescue of lysosome formation. If Pdpep1.3 exerts this effect through disruption of the a-syn-CHMP2B interaction, mutations that disrupt it should have a similar effect. Thus, a number of mutants of a-syn in the region of amino acid 103-114 were made and found that they reduced a-syn cytotoxicity as well as restored lysosomes as measured by LAMP1 levels (FIG. 10D). Moreover, a truncated version of A53T a-syn lacking amino acids 103-140 in neurons which was HA-tagged at its N-terminus was expressed. It was observed that, compared with Scramble1.3, Pdpep1.3 did not reduce levels of this truncated a-syn measured by HA immunostaining (FIG. 10E, 10F). Therefore, the a-syn-CHMP2B interaction is necessary for the a-syn lowering effects of Pdpep1.3.

The effect of Pdpep1.3 on CD63, a marker of MVBs early in the endolysosomal pathway (33) was then assessed. Expression of A53T a-syn was associated with a substantial loss of CD63, consistent with disruption of MVB formation and the endolysosomal pathway, as indicated by the LAMP1 decrease. Co-expression with Pdpep1.3 resulted in the return of CD63, indicating the peptide rescued MVB formation (FIG. 3G).

Next, it was sought to directly test whether Pdpep1.3 affects lysosomal function. To this end, a lysosomal flux assay in which a self quenched substrate taken up by cells demonstrates a fluorescence signal proportional to intracellular lysosomal activity as the substrate is degraded (48) was used. As expected, it was found that A53T a-syn co-expressed with Scramble1.3 reduced lysosomal flux, consistent with a disruption of lysosomal activity (FIG. 3H). However, co-expression with Pdpep1.3 returned lysosomal flux to baseline (FIG. 3H), suggesting rescue of lysosomal maintenance by ESCRT.

Finally, it was sought to distinguish whether the degradation pathway disrupted by a-syn and rescued by Pdpep1.3 is autophagy-related (i.e., the autophagy-lysosomal pathway) or involves direct degradation by the lysosome without autophagosome involvement. To this end, p62, a prototypical autophagy receptor (34) was knocked down, and it was found that Pdpep1.3's effect on cell viability was not significantly affected, suggesting Pdpep1.3 acts independently of the autophagy-lysosomal pathway (FIG. 3I, 7C). From the findings, it is inferred that the effect of Pdpep1.3 is instead mediated by the endolysosomal pathway. It is proposed that accumulation and oligomerization of a-syn lead to a feedback loop in which initial a-syn oligomers inhibit protein degradation by disrupting ESCRT (via CHMP2B binding); both MVB formation and lysosome maintenance are disrupted, thus decreasing lysosomal protein degradation (FIG. 3J). While a-syn can be cleared from the cell via a number of ways (35), lysosomes play a key role in a-syn clearance (36). Thus, disruption of lysosomal activity increases a-syn levels which, in turn, further exacerbates lysosomal dysfunction (37). It is proposed that this feedback cycle can be broken by Pdpep1.3, which disrupts the a-syn-CHMP2B interaction, releasing CHMP2B to restore lysosomal degradation of a-syn (FIG. 3J).

Pdpep1.3 Reduces a-Syn-Mediated Neurodegeneration In Vivo

Overexpression of WT or mutant a-syn in dopaminergic neurons causes degeneration of neurites and eventual loss of soma in several animal models (38). Such models were used to examine whether treatment with Pdpep1.3 could attenuate a-syn-mediated neurodegeneration in vivo.

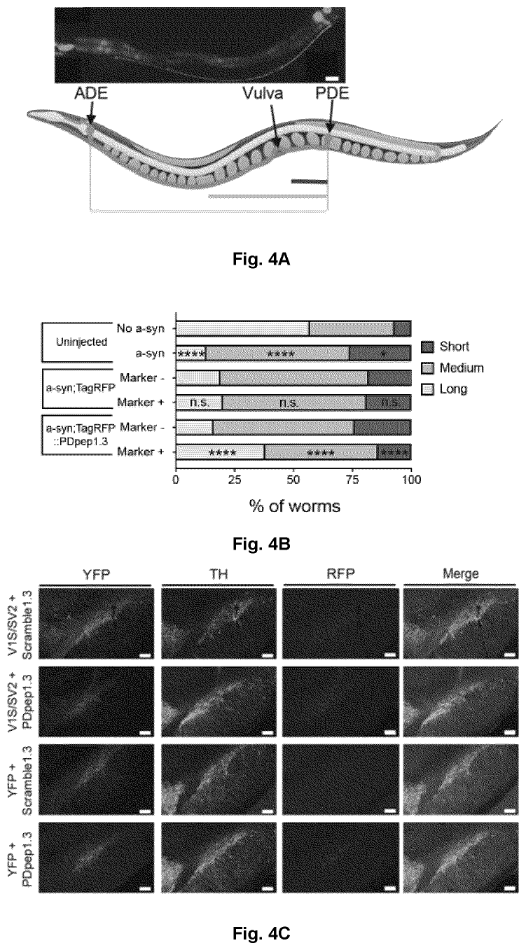

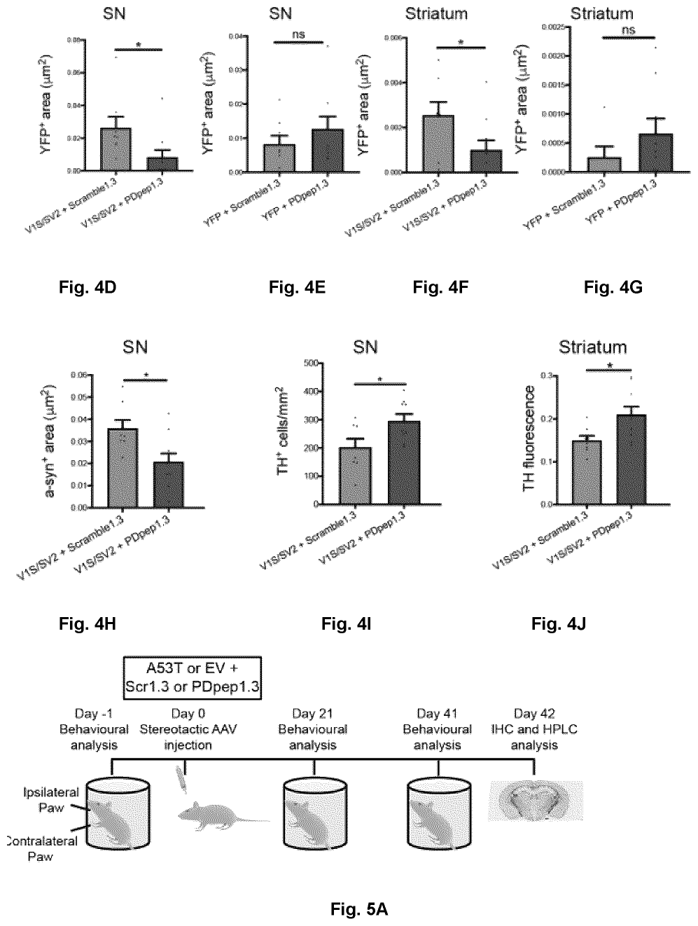

First, Pdpep1.3 in C. elegans (FIG. 4A) using a version of the peptide based on the nematode amino acid sequence of CHMP2B was tested. Overexpression of mutant A30P human a-syn resulted in significant neurite shortening of the posterior deirid (PDE) dopaminergic neurons, which was partially rescued by co-expression of RFP-tagged Pdpep1.3, but not RFP alone (FIG. 4B).

Second, the effect of Pdpep1.3 on a-syn oligomerization in an AAV vector-based rat model by directly co-injecting AAV-V1S and AAV-SV2 (a-syn fused to N- or C-terminal halves of YFP, respectively was determined; see above) in the substantia nigra (SN), as it was done previously (16), with AAVs that express Pdpep1.3 or Scramble1.3. At 6 weeks post-injection, a significant reduction in a-syn oligomer levels was observed, measured by YFP positive area, in dopaminergic neurons in the SN (FIG. 4C, 4D, 4E) and their axonal projections in the striatum (FIG. 4F, 4G) of rats that received Pdpep1.3 compared with Scramble1.3. When YFP fluorescence intensity in SN cells positive for both TH (i.e., dopaminergic neurons) and RFP (i.e., cells expressing peptide) was measured, it was also found that dopaminergic neurons expressing Pdpep1.3 had a lower mean fluorescence intensity (0.063±0.0167 relative fluorescence units (RFU)) than those expressing Scramble1.3 (0.154±0.034 RFU) (n=8 to 9 rats/group, one-way ANOVA, P<0.05). These findings with Pdpep1.3 were associated with a decrease in total a-syn positive area in the SN (FIG. 4H), as well as an increase in surviving dopaminergic neurons in the SN (FIG. 4C, 4I) and their terminals in the striatum (FIG. 4J, 11A), quantified using tyrosine hydroxylase (TH) as a dopaminergic marker.

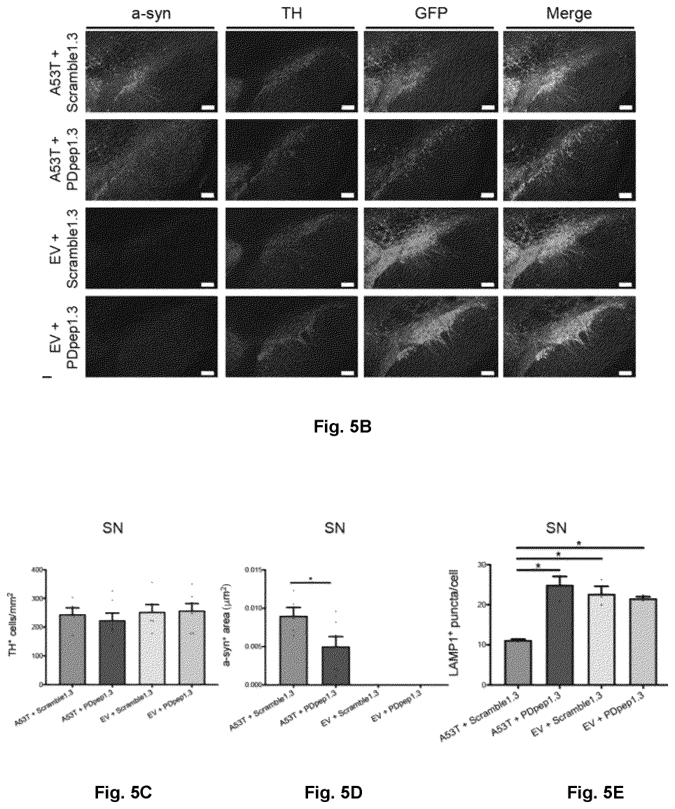

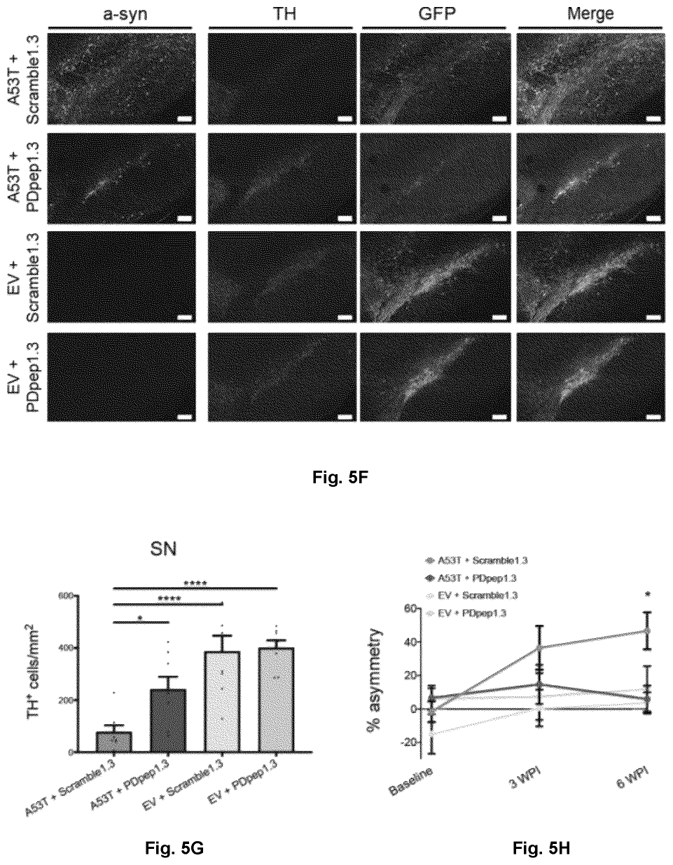

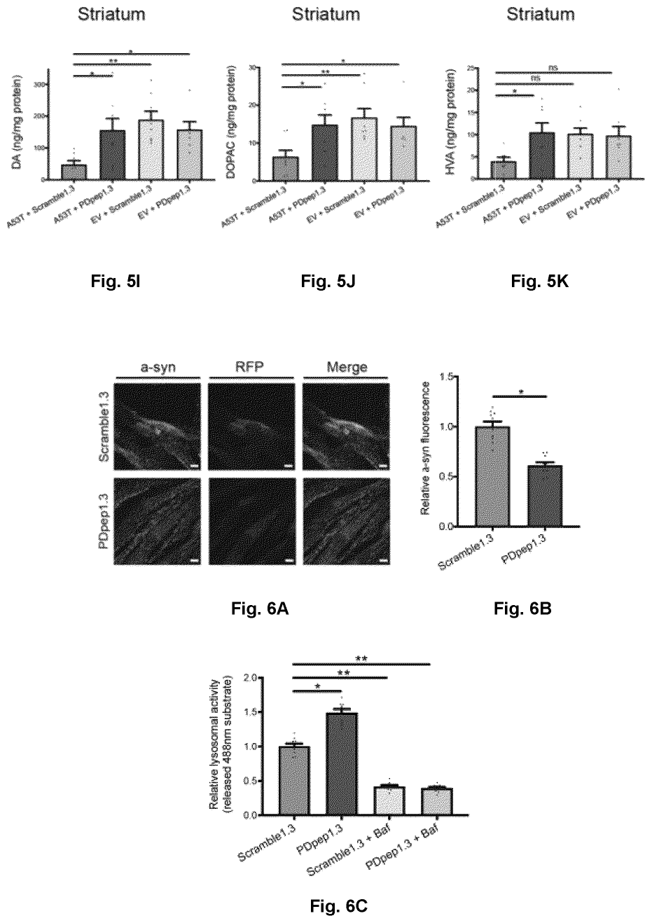

Third, a preclinical rat model of PD in which SN degeneration is induced by AAV vector-mediated expression of human mutant A53T a-syn (39-42) (FIG. 5A) was used. Since A53T a-syn overexpression results in substantial loss of SN neurons in this model, thus making it difficult to assess for reduction in a-syn levels not due to neuronal loss, A53T a-syn overexpression was initially limited by using a lower titer of AAV-A53T a-syn that did not result in overt neurodegeneration (43) (FIG. 5B, 5C). A significant reduction in total a-syn positive area in the SN of Pdpep1.3 treated animals compared with Scramble1.3 (FIG. 5B, 5D) was found. When a-syn fluorescence intensity in cells positive for both TH (i.e., dopaminergic neurons) and GFP (i.e., cells expressing peptide) were measured, it was also found that dopaminergic neurons expressing Pdpep1.3 had a lower mean fluorescence intensity (0.335±0.025 relative fluorescence units (RFU)) than those expressing Scramble1.3 (0.399±0.015 RFU) (n=5 to 7 rats/group, one-way ANOVA, P<0.05). Furthermore, overexpression of A53T a-syn was associated with a reduction in LAMP1-positive puncta in the SN which was restored with Pdpep1.3 (FIG. 5E, 11B). A higher titer of AAV-A53T a-syn in which animals receiving Scramble1.3 demonstrated significant dopaminergic cell death in the SN at 6 weeks post-injection (FIG. 5F, 5G) was then used. This neurodegeneration was mitigated by treatment with Pdpep1.3; the remaining number of surviving dopaminergic neurons was not different from animals injected with empty AAV vector instead of A53T a-syn and was significantly greater than treatment with Scramble1.3 (FIG. 5F, 5G). Forelimb asymmetry in the cylinder test is a behavioural impairment associated with SN degeneration which models bradykinesia, the cardinal motor abnormality in PD (43). Rats injected with A53T a-syn and Scramble1.3 displayed significant asymmetry in forelimb use at 6 weeks, whereas the behaviour of those injected with A53T a-syn and Pdpep1.3 was comparable to controls (FIG. 5H). Consistent with mitigation of dopaminergic neuron loss by Pdpep1.3, striatal levels of dopamine (FIG. 5I) and its metabolites, 3,4-dihyroxyphenylacetic acid (DOPAC) (FIG. 5J) and homovanillic acid (HVA) (FIG. 5K), were restored compared with the Scramble1.3 condition.

Pdpep1.3 Reduces a-Syn Accumulation in Human Cells with PD-Associated Mutations

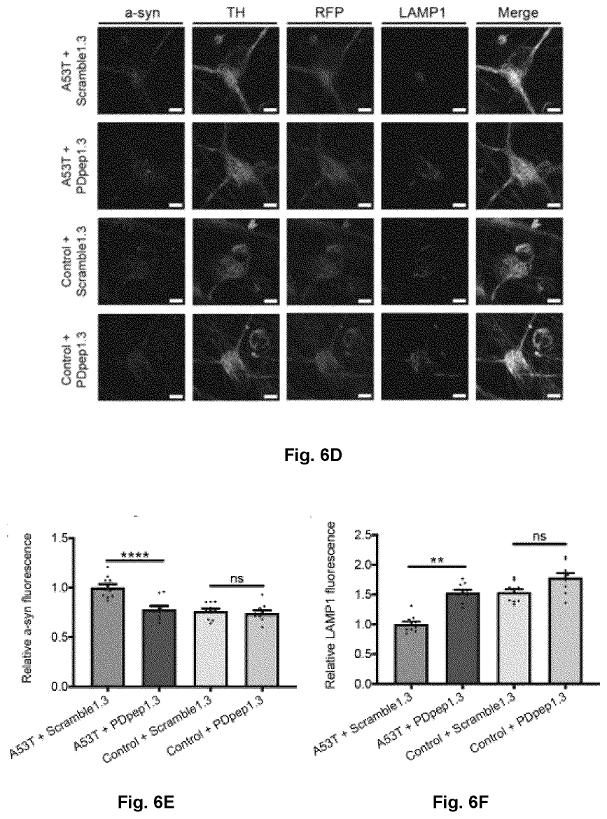

Finally, to investigate the effects of Pdpep1.3 on endogenous a-syn in disease-relevant human models, cells with a-syn gene (SNCA) mutations known to cause PD were used. An autosomal dominant form of PD is caused by triplication mutation of SNCA with doubling in the effective load of the WT gene peripherally and in the brain (44). Skin fibroblasts from a PD patient with SNCA triplication and transduced them with AAVs expressing RFP-tagged peptides were cultured. It was found that Pdpep1.3 reduced a-syn fluorescence compared with Scramble1.3 control (FIG. 6A, 6B). This reduction in a-syn levels was associated with increased lysosomal activity as measured using the lysosomal flux assay described above (FIG. 6C). Dopaminergic neurons from induced pluripotent stem cells (iPSCs) harbouring the A53T a-syn mutation and from isogenic iPSCs in which this mutation was absent were also derived. iPSC-derived dopaminergic neurons from people with genetic or idiopathic PD have been observed to accumulate a-syn protein (45)(46)(47). Under control conditions (i.e., expression of Scramble1.3 peptide), it was also observed a-syn accumulation in A53T a-syn iPSC-derived dopaminergic neurons when compared with isogenic control neurons (FIG. 6D, 6E). Further, LAMP1 levels were reduced in A53T a-syn neurons (FIG. 6D, 6F). It was found that expression of Pdpep1.3 in A53T a-syn neurons returned both a-syn and LAMP1 levels to those of controls (FIG. 6D, 6E, 6F). Taken together, these data suggest that, in addition to its neuroprotective effects in animal models, Pdpep1.3 is efficacious at reducing a-syn levels and restoring the endolysosomal pathway in human PD models.

Further details of the methods and materials are provided in Example 3.

The results show that a-syn inhibits the endolysosomal pathway via interaction of its C-terminal region with CHMP2B. It is proposed that a-syn oligomers are instrumental in this inhibition since the C-terminal regions of multiple a-syn proteins are exposed in oligomers, thus avidity will lead to enhanced affinity of the a-syn-CHMP2B interaction. Moreover, this reduced lysosomal function will likely lead to reduced degradation of a-syn itself, thus resulting in further accumulation, which in turn leads to further lysosomal disruption. The findings thus illustrate a potential mechanism by which a-syn oligomerization can lead to severe failure of the cellular machinery.

The optimized peptide derived from the proteomic screens, Pdpep1.3, directly disrupts the a-syn-CHMP2B interaction, breaking the feedback loop and thereby restoring ESCRT function and lysosomal degradation. The net effect is an increase in lysosomal activity in neurons, increasing the clearance of a-syn, including a-syn oligomers. The effectiveness of Pdpep1.3 in promoting clearance of overexpressed a-syn both in vitro and in vivo has been demonstrated, and it has been shown that expression of Pdpep1.3 can reduce dopaminergic cell death in preclinical models of PD. also It has also been shown Pdpep1.3 to be efficacious at inhibiting a-syn accumulation and restoring the endolysosomal pathway in human PD models. This has potential important therapeutic implications as a peptide, such as Pdpep1.3, that facilitates a-syn degradation to reduce overall a-syn protein levels will circumvent many of the challenges faced by current approaches which depend on targeting specific a-syn conformations. Thus, targeting this novel pathogenic interaction between a-syn and CHMP2B may hold promise as a disease-modifying therapeutic strategy for the treatment of PD.

### Example 2

An optimized library based on the Pdpep1.3 peptide was designed in order to identify variants with higher activity. It is demonstrated that Pdpep1.3 peptide binds to CHMP2B and inhibits the CHMP2B-VPS4 interaction. Consequently, a I approach by building a combinatorial library of single mutants, and double mutants in positions I, i+4 was adopted. Also, extended and shortest variants of the Pdpep1.4 peptide were included by including amino acids of CHMP2B upstream and downstream of the natural sequence. Parent, WT asyn and A53T cell lines were used to screen for peptides that rescue cell viability, and the top 10 peptides are displayed in FIG. 12A. A handful of peptides showed better effect on cell viability than Pdpep1.3 (FIGS. 12B and C) and were selected for further experiments.

One approach to overcome the typical limitations of peptide cellular uptake, half-life in blood, and BBB crossing efficiency is stapling the peptide by covalently linking the sidechains of two amino acids using an hexafluorobenzene, thereby forming a peptide macrocycle. Molecular dynamic simulations were performed to measure the stability of 5 different alternatives. The peptide structure was based on the C-terminal fragment of CHMP2B on the PDB 2JQK. The heaxaflurobenze was parametrized using amber tools (antechamber, and xleap) and gaussian. Each structure model was explicitly solvated by TIP3P water molecules in truncated octahedral periodic boundary conditions, and sodium counter ions were added for overall charge neutrality. Then, each system was minimized and equilibrated and a total of 100 ns of simulated were generated. Three out of the five were selected based on the performance during the simulations and the information about the potential hotspots in the sequence inferred and collected from all the experiments. The best candidates were DEEYCRQWKALC (SEQ ID NO: 35), DIEICFQLKALC (SEQ ID NO: 36) and DEEICRQLDALC (SEQ ID NO: 37) (Pdpep1.4 variants) (the C marks the residues modified for stapling-peptides to be chemically stapled are modified to introduce Cysteines at the locations to be stapled and these Cysteines are used for the chemical stapling). Optimized peptides DEEYCRQWKALC (SEQ ID NO: 35), DIEICFQLKALC (SEQ ID NO: 36) and DEEICRQLDALC (SEQ ID NO: 37) (Pdpep1.4 variants) showed increase in A53T cell viability (FIG. 13A) and significant reduction in a-syn aggregation from cells stably expressing split luciferase a-synuclein constructs (FIG. 13B).

Affinity measurements revealed that optimized macrocycles bind to truncated CHMP2B (1-199) as well as full length CHMP2B (FIG. 3). Pdpep1.4 variant DEEICRQLDALC (SEQ ID NO: 37) had the most affinity to truncated CHMP2B (1-199) (Kd=60 nM) and full length CHMP2B (Kd=0.6 uM). Taken together, these results imply that macrocycles protect against a-syn cytotoxicity by targeting interactions of CHMP2B.

### Example 3

### Materials and Methods

### Next-Generation Sequencing Preprocessing

PCR primers that included the Illumina adaptor sequences were used to amplify peptide coding sequences recovered from selection and cell sorting. Results were demultiplexed, and peptide counts were tallied. After demultiplexing, only reads with an average quality Phred score of >30 were selected, and frequencies were calculated. Finally, the reads were then normalized to the total number of peptides read for that sample population, and a scalar factor was applied. In parallel, ˜25 individual colonies were Sanger sequenced to compare to the NGS data.

### Determination of Target Interactions

The identification of potential targets for the active peptides was conducted by mapping the sequences to the interacting interfaces of protein-protein interactions in the PDB. Multiple sequence mismatches were allowed, and a maximum of 3 mismatches were applied. The mapping complexes were ranked by sequence identity and the number of GO terms enriched in PD were annotated for the proteins involved in the PPI (Table 1). The enrichment term analysis was performed using DAVID based on the list of 330 genes annotated by the Parkinson's Disease Gene Ontology Annotation Institute at University College London. Next, how many of the Parkinson's standard GO terms were shared by the mapped proteins on each structure complex were counted. This record was used to rank and prioritize the matches. After visual inspection of a list of ten hits by discarding crystal packing contacts, and non-significant matches, the mapping of IPIQLKA (SEQ ID NO: 1) to the structure of the complex of a C-terminal fragment of CHMP2B binding to the MIT domain of VPS4B (PDB 2JQK) was selected. Both proteins belong to the ESCRT-III complex and play a vital role in vesicular body formation.

### Purification of CHMP2B

Overexpress® C41 E. coli cells were transformed by heat shock with 100 ng of pET-DEST42 CHMP2B vector and plated in LB plates containing 50 μg/ml of Carbenicillin for overnight incubation at 37° C. Transformed cells were grown in 500 ml of 2×YT media at 37° C. shaking at 220 rpm in a baffled 2 L flask. Cultures were grown until an OD of 0.6 was reached and then induced with 0.5 mM IPTG. After induction, cells were left shaking at 37° C. for a further 3 h at 220 rpm. Faster expression demonstrated reduced levels of non-specific truncations compared to overnight incubations at lower temperature. Cultures were pelleted at 3000×g and pellets were resuspended in 20 ml of BugBuster® Master Mix per 500 ml culture. The lysis reactions were incubated for 20 min at 4° C. mixing in a tube rotator. CHMP2B constructs expressed as inclusion bodies were insoluble in the BugBuster mix. The lysates were spun for 20 min at 3000×g to separate the inclusion bodies from the rest of the cell debris. Inclusion bodies were resuspended in 35 ml of 10 mM Phosphate pH 7.4, 150 mM NaCl, 6 M Guanidine HCl and spun at 34000×g at 4° C. for 20 min to remove lipidic contaminants. The supernatant was mixed with 5 ml of Ni-NTA resin in batch and incubated for 20 min in a tube rotator. Ni-NTA resins were pelleted by centrifugation at 270×g for 5 min and the supernatant was removed. The resins were washed three times with 10 mM Phosphate pH 7.4, 150 mM NaCl, 6 M Guanidine HCl, 30 mM Imidazole and proteins were eluted in 10 mM Phosphate pH 7.4, 150 mM NaCl, 6M Guanidine HCl, 500 mM Imidazole to a total volume of 10 ml. The eluted samples were dialysed overnight in 5 L of 50 mM Na Acetate pH 5.5 and 0.5 mM TCEP at 4° C. with a 10 kDa cutoff dialysis membrane. CHMP2B was further purified by Size exclusion chromatography with a HiLoad Superdex 16/60 S200 in an AKTA purifier. The column was pre-equilibrated in fresh 50 mM Na Acetate pH 5.5 and 0.5 mM TCEP, as CHMP2B displays greatly improved solubility in slightly acidic pH. CHMP2B eluted at the expected volume for a 26 kDa monomer. Samples were concentrated in a Amicon Ultra-15 spin concentrator to 0.5 mg/ml and stored at −80° C. Sample purity was confirmed by SDS-PAGE and protein identity was validated by ESI Mass Spectrometry.

### Purification of VPS4B

VPS4B was expressed as a His-tagged Sumo construct in a pRSet B vector. Overexpress® C41 E. coli cells were transformed by heat shock with 100 ng of vector and cultures were grown as described for CHMP2B. Cultures were induced with 0.5 mM IPTG and incubated overnight at 20° C. Cells were pelleted as described and resuspended in PBS with a cOmplete Mini, EDTA-free protease inhibitor tablet. Cells were lysed by sonication in a Branson Digital Sonifier at 20% amplitude for 5 minutes in 10 s intervals between on and off sonication. The homogenized sample was spun at 34000×g at 4° C. for 20 min to remove insoluble contents. The soluble supernatant was incubated with 5 ml of Ni-NTA resin in batch for 20 min at 4° C. in a tube rotator. The resins were washed three times in PBS with 30 mM Imizadole and eluted in PBS with 300 mM imidazole. Imidazole was removed by dialyzing twice into 5 L of PBS.

### Cloning of the Lentiviral Library

To identify peptide inhibitors of protein-protein interaction, a previously designed human peptide library containing 50,549 heptamer C-terminal sequences, corresponding to 75,797 proteins, including isoforms and cleaved sequences was used. The oligonucleotide libraries were amplified and cloned into pLJM1 nGFP vector as previously described (12).

### Cell Lines and Reagents

HEK293T cells, tet-off split luciferase a-syn cells, tet-off parent cells and SH-SY5Y cell lines were maintained in DMEM (ATCC) supplemented with 10% FBS and 1% pen/strep/glutamine, and the appropriate selection antibiotics when required. tet-off split luciferase a-syn cells and tet-off parent cells were kept with doxycycline at 1 ng/mL for inhibition of gene expression. SNCA triplication fibroblasts were obtained from NINDS (National Institute of Neurological Disorders and Stroke) and maintained in DMEM supplemented with 10% FBS and 1% pen/strep/glutamine. HA antibodies were obtained from Santa Cruz (7392) and Flag antibodies were purchased from Sigma (A8592). GFP antibodies were purchased from Abcam (ab290).

### Lentiviral Delivery and Cell Screens

Lentiviruses were made in a 15 cm dish format by transfecting packaging cells (293T) with a three-plasmid system as previously described. Viral transduction of HEK293T cells, tet-off split luciferase a-syn cells, tet-off parent cells, and SH-SY5Y cells were performed with a multiplicity of infection of 0.3 (MOI=0.3). Infected cells were selected in puromycin-containing medium to eliminate uninfected cells and three aliquots of cells were collected for sampling of the initial (TO) cell population. Cells were treated with MG132 at concentrations of 0 μM, 10 μM, 25 μM or 50 μM. For the cell viability screen, surviving cells were collected and gDNA was extracted for identification of peptide inhibitors of cell toxicity. For the inhibition of a-syn aggregation screen, split YFP-a-syn constructs were transfected following stable expression of the peptides. Cells were sorted based on their GFP fluorescence intensities with a FACSVantage SE cell sorter (BD Bioscience).

### Fluorescence-Activated Cell Analysis and Sorting

Cells were harvested by trypsin treatment and centrifuged at 500×g for 5 minutes. The pellet was resuspended in ice-cold PBS and centrifuged again. The pellet was then resuspended at a concentration of 4×10⁶ cells/ml in the sorting buffer, which is PBS containing 100 Kunitz Dnase l/ml, 10 μg/ml propidium iodide (Sigma) and 2% FBS. The sorting solution was also supplemented with either 10 μM forskolin, 100 μM 5,6-Dichlorobenzimidazole riboside (DRB, Sigma), 10 μM forskolin (Sigma) and 100 μM DRB or DMSO, as a control. The cells were then sent through a 40 μm filter to remove large clumps and loaded into either a FACScan Flow Cytometer (BD Bioscience) for cell analysis or a FACSVantage SE cell sorter (BD Bioscience) for cell sorting. The cells with positive propidium iodide staining (i.e., dead cells) were first eliminated from the analysis or sorting pool. For cell sorting, the desired population, either the most or least bright EGFP-positive cells, according to the purpose of the experiments (see Results), was sorted into either 15 ml conical tubes or 96-well plates, which both contained complete DMEM culture media.

### Genomic DNA Preparation and Illumina Sample Preparation

Genomic DNA (gDNA) from peptide expressing cells at different time-points was extracted using QIAamp DNA Blood Mini Kit. PCR amplifications of peptides from gDNA in parallel with the lentiviral plasmid library (naïve library) were performed using indexed Illumina PCR primers to incorporate both the Illumina adapter sequences and indexing sequences. Each 50 μl reaction contained 3.2 μg of template, 2×PCR buffer, 2× enhancer solution, 300 μM each dNTP, 900 nM each of Adapter A (5′-AATGATACGGCGACCACCGAAATG-GACTATCATATGCTTACCGTAACTTGAA-3′) (SEQ ID NO: 42) and Adapter B (5′-CAAGCAGAA-GACGGCATACGATGTGGATGAATACTGCCATTTGTCTCGAGGTC-3′) (SEQ ID NO: 43), 1 mM MgSO4, 3.75 units of Platinum Pfx polymerase, and water to 50 μl. The PCR reaction was performed by denaturing at 94° C. for 5 minutes, followed by cycling (94° C. for 30 seconds, 65° C. for 30 seconds, 68° C. for 30 seconds)×28 cycles, 68° C. for 5 minutes, then cooling to 4° C. The resulting 244 bp product was purified by electrophoresis in 2% agarose followed by gel extraction. Peptide libraries were quantified using Quant-It assay (Invitrogen) and pooled. The insert size of the pooled library was confirmed on an Agilent Bioanalyzer High Sensitivity DNA chip (Agilent Technologies), and the size corrected concentration was determined with RT-qPCR (KAPA biosystems Illumina standards). 11.4 pM of peptide library and 0.6 μM of PhiX control library (Illumina) were denatured and loaded on a HiSeq 2000 V3 150 cycle sequencing kit, with a read length of 150 bp.

### Validation of Individual Peptides

Oligonucleotides encoding the specific peptides were synthesized and individually cloned into the pLJM1 nGFP lentiviral vector. Cells were infected with individual constructs, and cell viability was assessed using Cell Titer-Glo Luminescent assay (Promega) at 72 h post infection.

### Cell Titer-Glo Luminescent Cell Viability Assay

Cells were trypsinized from subconfluent cultures as described earlier, suspended in culture media, and then seeded into triplicate wells of a 96-well plate (100 μl well/1) at a density of 1.5×10⁴ cells per well at standard culture conditions of 5% CO2 in air at 37° C. Cells were infected with lentivirus expressing peptide at an MOI of 5 for 72 h or transfected with plasmid for 72 h. Cell Titer-Glo reagent was added to each well (30 μL), according to the manufacturer's protocol and optical density of the plate was measured at 540 and 630 nm with a standard spectrophotometer.

### Immunoblotting

Infected or transfected cells were scraped from 6-well dishes and lysed with lysis buffer (50 mM Tris-HCl pH7.4, 1% Nonidet P-40, 150 mM NaCl, 1 mM EDTA, 1× protease inhibitor mixture (Sigma)) for 30 min at 4° C. The insoluble pellet was removed following a 10,000 rpm spin for 5 minutes at 4° C. Lysates were analyzed by SDS-PAGE/immunoblot using 4-20% Mini-PROTEAN Tris-glycine gels (Bio-Rad) transferred to PVDF membranes and blocked in 5% milk containing PBS-Tween-20 (0.1%) for 1 h. PVDF membranes were then incubated with specified primary antibodies followed by incubation with horseradish peroxidase-conjugated secondary antibodies (Santa Cruz Biotechnology) and detected using enhanced chemiluminescence (GE Healthcare).

Infected neurons were scraped from 6-well dishes and lysed with RIPA buffer containing protease inhibitor cocktail (Roche). The Triton X-100 soluble fraction was then separated from the insoluble pellet by centrifugation. Protein concentration was quantified using the DC protein assay (BioRad). For each condition, 20 μg of protein lysate was run on 4-15% acrylamide gels (BioRad) and subsequently transferred onto a polyvinylidene difluoride (PVDF) membrane. Blots were blocked with 5% skim milk in TBS+0.01% Tween-20 (TBS-T) for 1 h prior to incubation with primary antibody overnight at 4° C. Blots were subsequently washed 3 times in TBS-T for 10 minutes per wash, incubated in species specific secondary antibody for 1 h at 21° C., washed again, and then developed using ECL immunoblotting substrate (Pierce) and visualized on HyBlot CL autoradiographic film (Denville Scientific).

### Flag Co-Immunoprecipitation

HEK293T cells were co-transfected with Flag-tagged target protein, HA-tagged source protein, and GFP-tagged peptide or GFP. Cells were lysed 48 h after transfections with radioimmune precipitation assay buffer (50 mM Tris-HCl pH7.4, 1% Nonidet P-40, 150 mM NaCl, 1 mM EDTA, 10 mM Na3VO4, 10 mM sodium pyrophosphate, 25 mM NaF, 1× protease inhibitor mixture (Sigma)) for 30 min at 4° C. and coimmunoprecipitated with Flag beads (Clontech). The resulting immunocomplexes were analyzed by immunoblot using the appropriate antibodies. Protein samples were separated using 4-20% Mini-PROTEAN Tris-glycine gels (Bio-Rad) transferred to PVDF membranes and blocked in 5% milk containing PBS-Tween-20 (0.1%) for 1 h. PVDF membranes were then incubated with specified primary antibodies followed by incubation with horseradish peroxidase-conjugated secondary antibodies (Santa Cruz Biotechnology) and detected using enhanced chemiluminescence (GE Healthcare).

Purification of a-syn

Glycerol stocks were plated on LB plates containing 50 μg/ml of kanamycin for overnight incubation at 37° C. Bacterial cultures were grown in 500 ml of LB media at 37° C. with shaking at 220 rpm in a baffled 2 L flask. Cultures were grown until an OD of 0.6 was reached and then induced with 0.5 mM IPTG. After induction, cells were incubated at 37° C. for a further 2.5 h shaking at 220 rpm. Cells were harvested by splitting the 500 ml culture into two 250 ml conical tubes and spinning down at 4000× g for 15 min at 4° C. Cells were re-suspended in lysis buffer (50 mM KH₂PO₄, 400 mM NaCl, 100 mM KCl, 30 mM Imidazole, 10% v/v glycerol, 0.5% v/v Triton X-100) and lysed by 3 cycles of flash freezing on an ethanol/dry-ice mix for 12 mins, followed by heating in a water bath for 12 mins. The resultant mixture was spun down at 4000× g for 15 min at 4° C. and the supernatant was added directly to a His-spin trap (GE Healthcare) as per the manufacturer's instructions. The eluted product was dialyzed against PBS overnight, aliquoted at a concentration of 1 mg/ml and stored at −80° C. until ready for use.

### Fluorescence Polarization Assays

Fluorescence polarization assays were carried out in 384-well black non-binding plates (Greiner 781906) in a Pherastar plate reader (BMG) with a Fluorescence Polarization Module 485-520-520. All peptides were synthesized by LifeTein with N-terminal FITC moieties. Binding assays were performed in 50 mM Na Phosphate pH 5.5, 0.5 mM TCEP, 0.005% Triton-X100 (2) or in 20 mM Na Acetate pH 5.5, 0.5 mM TCEP, 0.005% Triton-X100 as indicated. FITC-labelled peptides were kept at a constant 50 nM concentration and CHMP2B was serially diluted 1:1 starting from 30 μM (0.5 mg/ml).

The a-syn competition assay had a constant 50 nM concentration of FITC-Pdpep1.3 peptide and 1 μM of CHMP2B; a-syn was serially diluted 1:1 starting from 30 μM. Plates were incubated for 30 min at room temperature before reading. Raw FP data in millipolarization (mp) units was fitted to the following equation in Graphpad PRISM 8:

$$Y = \frac{B max \cdot X}{K d + X} + Background$$

where X is the concentration of CHMP2B. The equation was fitted with and without Hill value (h) to check for h˜1. As the FITC-labelled peptide is at a concentration under 10 times the K_{d}, total CHMP2B concentration can be assumed to approximate free CHMP2B concentration. Bmax represents the polarization units in maximum association and Background represents the polarization units in absence of CHMP2B. Y is in milli-polarization units (mp).

### Luciferase Assay

tet-off cells stably expressing split luciferase a-syn constructs were trypsinized from subconfluent culture and seeded in a 96-well plate at a density of 15,000 cells per well. Cells were incubated overnight at 37° C. in 5% CO₂. Cells were transfected with GFP-peptide plasmids. After 6 h of incubation, 20 μL of cell medium was transferred to a black flat-bottomed 96-well plate. 50 μL of Working solution (Pierce Gaussia-Firefly Luciferase Dual Assay Kit, Thermo Scientific #16181) was added into each well containing cell medium. Immediately after adding the reagent, samples were read using a luminometer with a 480 nm filter.

### Lysosome Activity Assay

The Lysosome Intracellular Activity Assay kit was purchased from Abcam and used per manufacturer's instructions (ab234622). Briefly, cell medium was replaced with fresh medium containing DMSO, Leupeptin, or Bafilomycin A1 and incubated for 1 h at 37° C. with 5% CO₂. Self-quenched substrate was added into each condition and incubated for 1 h (HEK293 cells) or overnight (SNCA triplication fibroblasts) at 37° C. with 5% CO₂. After incubation, cells were washed twice in ice-cold 1× assay buffer containing DMSO, Leupeptin, or Bafilomycin A1. For HEK293 cells, cells were re-suspended in PBS containing DMSO or Leupeptin and analyzed by FACS. For fibroblasts, cells were incubated in PBS containing DMSO or Bafilomycin A1 and imaged by confocal microscopy.

### Adeno-Associated Viruses

Adeno-associated virus (AAV) of a % serotype was used to express A53T a-syn (AAV-A53T), a truncated version of A53T a-syn lacking amino acids 103-140 (Aa-syn(103-140)), and WT a-syn, fused to either the N-terminus half of YFP (AAV-V1S) or the C-terminus half of YFP (AAV-SV2), all under the control of the CAG promoter, a hybrid of the chicken beta actin (CBA) promoter fused with the cytomegalovirus (CMV) immediate early enhancer sequence (GeneDetect Ltd.), as previously described (3). An AAV1/2 vector lacking the A53T a-syn open reading frame was used as an empty vector control (AAV-Empty) for A53T a-syn experiments and an AAV1/2 vector expressing full length YFP was used as a control for experiments measuring a-syn oligomer formation.

### Primary Neuron Culture and AAV Transduction

Pregnant rats (E17) of the Sprague-Dawley strain were purchased from Envigo. Embryos were surgically removed from the mothers and cortices dissected in Hanks Balanced salt solution (Gibco). The meninges were removed, and cells were dissociated using a papain dissociation system (Worthington) before being resuspended in Neurobasal medium A supplemented with antibiotic-antimycotic solution (Gibco), L-glutamine substitute (GlutaMAX™; Gibco) and factor B27 (Gibco). Cells were plated on poly-D-lysine coated glass coverslips at a density of 5×10⁵ cells/well, or on poly-D-lysine coated 6-well cell culture plates at a density of 2×10⁶ cells/well, and incubated at 37° C. in 5% CO₂ with half media changes every 3 days. Cells were transduced with AAVs 2 days post-isolation at a multiplicity of infection (MOI) of 3000. Media containing AAV vectors were removed after 72 h and cells were fixed with 4% PFA for immunofluorescence staining or lysed for immunoblotting at 8 days post-isolation.

Human iPSC-Derived Dopaminergic Neuron Culture

iCell Dopa neurons (A53T a-syn mutant and isogenic control) were purchased from Fujifilm Cellular Dynamics (Madison, WI, USA) and cultured as per manufacturer's instructions. Briefly, cells were plated in complete maintenance medium provided by the manufacturer on poly-L-ornithine/laminin coated plates (ibidi). Cells recovered from thawing for 24 h and then were transduced with AAV-Scramble1.3-RFP or AAV-Pdpep1.3-RFP for 48 h at which time media was completely changed. Cells were fixed using 4% PFA at 7 days post-plating.

### Immunofluorescence Staining of Cultured Cells

After fixation, cells were permeabilized with 0.2% Triton X-100 for 15 min, washed 3 times with PBS and then incubated with blocking solution (1% BSA, 22.52 mg/mL glycine, 0.1% Tween-20 in PBS) for 1 h. Primary antibodies were diluted in blocking solution and incubated overnight at 4° C. Following three washes with PBS, cells were next incubated with fluorescent secondary antibodies diluted in blocking solution for 1 h at room temperature. Following another 3 PBS washes, nuclei were counterstained with DAPI (ThermoFisher) and then coverslips were mounted on slides using fluorescence mounting medium (DAKO) and sealed using clear nail varnish.

C. elegans Strains

C. elegans strains were grown and maintained under standard conditions at 22-23° (4). BZ555 [dat-1p::gfp] was obtained from the C. elegans Genetics Center (CGC; University of Minnesota, St Paul, MN, USA), TWH1 ([dat-1p::a-syn(A30P), ges-1p::DsRed]; [dat-1p::gfp]) was obtained by crossing BZ555 with A30P a-syn transgenic animals (kindly provided by Dr. Takeshi Iwatsubo, University of Tokyo) (5).

C. elegans Neurite Length Assay

The peptide construct (pPD97.78_osm-6p_TagRFP_wpeptide) was obtained by subcloning the following fragments into NheI and SpeI sites of pPD97.78 (A. Fire): NheI-AgeI osm-1p fragment (2.4 kb) obtained by PCR using primer set 5′-catccgctagcggatcccatggccagtggaatcacc-3′ (SEQ ID NO: 44) and 5′-ccataccggtagatgtatactaatgaaggtaatagcttgaaagag-3′ (SEQ ID NO: 45) and N2 genomic DNA as a template, AgeI-EcoRI TagRFP fragment obtained by PCR using primer set 5′-gttgaccggtATGGTGTCTAAGGGCGAAGAGCTG-3′ (SEQ ID NO: 46) and 5′-ggcagaattcgaATTAAGTTTGTGCCCCAGTTTGCTAGG-3′ (SEQ ID NO: 47), EcoRI-SmaI peptide (FEELEAQLARLR (SEQ ID NO: 48)) fragment digested from CMV_FDDLEAQLARLR_worm, SmaI-SpeI unc-54 3′-UTR fragment digested from pPD95.75 (Fire vector). The TagRFP control construct was obtained by replacing the EcoRI-SmaI peptide fragment and SmaI-SpeI unc-54 3′-UTR fragment from pPD97.78_osm-6p_TagRFP_wpeptide, to EcoRI-SpeI unc-54 3′-UTR fragment digested from pPD95.75 (6).

Each plasmid (40 ng/μl) was injected with a co-injection marker, sur-5p::mCherry into TWH1. The animals carrying the extrachromosomal arrays were transferred to new plates, and their progenies were analyzed at the adult stage under a widefield microscope (Zeiss AxioObserver). Pearson Chi-Square test for pairwise comparison of neurite length frequencies was used for statistical analysis.

### Rats

Adult female Sprague-Dawley rats (250-280 g; Envigo) were pair-housed in cages with wood bedding and had access to food and water ad libitum. The animal colony was maintained in a regular 12-h light/dark cycle. All procedures were approved by the University Health Network Animal Care Committee in accordance with guidelines and regulations set by the Canadian Council on Animal Care.

### Stereotactic Surgery

Animals were secured in a stereotactic frame under isoflurane/oxygen anaesthesia and ketoprofen (5 mg/kg) analgesia. The surgical site was shaved and sterilized with iodine/betadine prior to making a 2 cm incision along the midline. The scalp was exposed and a unilateral injection targeting the SN was performed at coordinates AP−5.2 mm, ML−2 mm and DV−7.4 mm with respect to the bregma as a point of reference. For each animal, a total volume of 2 μl of virus was injected at a rate of 0.5 μl/min using a microinjection pump and 10 μl Hamilton syringe with a 26-gauge needle. For A53T groups, 1 μl (low dose) or 1.34 μl (high dose) of AAV1/2-A53T a-syn (5.1×10¹² genomic particles/ml), 0.14 μl of AAV1/2-Pdpep1.3-GFP or scramble1.3-GFP (5.1×10¹² genomic particles/ml) and 0.86 μl or 0.52 μl of sterile PBS was injected; for EV groups, 1 μl (low dose) or 1.34 μl (high dose) of AAV1/2-EV (5.1×10¹² genomic particles/ml) replaced AAV1/2-A53T. For V1S+SV2 groups, 0.58 μl of AAV1/2-V1S (1.1×10¹² genomic particles/ml), 0.58 μl of AAV1/2-SV2 (1.1×10¹² genomic particles/ml), 0.14 μl of AAV1/2-Pdpep1.3-RFP or scramble1.3-RFP (5.1×10¹² genomic particles/ml) and 0.7 μl of sterile PBS was injected; for the YFP groups, 1.16 μl of AAV1/2-YFP (1.1×10¹² genomic particles/ml), 0.14 μl of AAV1/2-Pdpep1.3-RFP or scramble1.3-RFP (5.1×10¹² genomic particles/ml) and 0.7 μl of sterile PBS was injected. At the end of virus injection, the needle remained in place for 5 minutes before gradual removal.

### Cylinder Test

Spontaneous forepaw use was evaluated using the cylinder test 1 day prior to stereotactic AAV injection, at 21 days post-injection and at 41 days post-injection. Following overnight food restriction, individual rats with right paws marked black were placed into a glass cylinder in front of two mirrors and videos recorded. An observer blinded to treatment conditions later scored the videos by recording whether animals used their left or right forepaw to touch the inner glass surface upon rearing. A total of 5 min of video recording was scored and a minimum of 10 total touches was required for data inclusion (61).

### Brain Tissue Preparation

Animals were uthanized by transcardial perfusion with heparinised saline under isoflurane/oxygen anaesthesia. Brains were then removed and the ventral part, including the ventral striatum, was snap frozen in liquid dry ice-cooled isopentane. A single 1 mm thick section of the ventral striatum was immediately cut, using a matrix, for high-performance liquid chromatography (HPLC) analysis of biogenic amines. The dorsal part, including the dorsal striatum, STN, and the SN, was immersion-fixed in 4% paraformaldehyde in 0.1M PBS for 2 days and cryo-protected in 30% sucrose in 0.1M PBS solution for another 3 days until the brains sank. For immunofluorescent staining, 40 μm coronal cryosections were then prepared using a sliding microtome (Leica Microsystems Inc.) and 6 series of sections were stored in cryoprotectant (30% glycerol, 30% ethylene glycol, 40% PBS) at −20° C. until use.

### Immunofluorescence Staining of Brain Cryosections

Immunofluorescence staining for a-syn, tyrosine hydroxylase and LAMP1 was performed by washing free-floating sections with PBS-T (0.2% Triton X-100) three times for 10 min each at room temperature. Sections were then incubated in blocking solution (1% BSA, 10% normal goat serum in PBS-T) for 1 h. After blocking, sections were incubated with primary antibodies in antibody solution (2% normal goat serum in PBS-T) overnight at RT. Sections were then washed in PBS and incubated with secondary antibodies diluted in antibody solution for 1 h in the dark at room temperature. Sections were then mounted onto glass slides and allowed to dry and then fluorescence mounting medium (DAKO) was applied followed by cover slips.

### Image Acquisition and Analysis

Confocal images of immunofluorescent staining were acquired with a Zeiss LSM880 confocal microscope equipped with 405, 488, 555, and 639 nm laser lines. All images were taken within the linear range at constant gain and pinhole settings at optimal resolution settings determined by the software. For primary cortical neurons, the software was programmed to acquire an image every 1 μm for a total of 11 μm, capturing all of the neurons visible in the z-plane in each field of view using a 63× objective. For animal experiments, the whole midbrain or striatum regions were imaged using a 10× objective. Ten serial coronal midbrain sections were imaged per animal, separated by 240 μm intervals. 3-6 images of the striatum per animal were acquired and a representative image of a single coronal section present in all sets was chosen for analysis, based on anatomical features.

Confocal images of immunofluorescent staining of midbrain and striatal sections were processed using HALO software (Indica Labs), which is a well validated tool for automatic quantification of neurons in brain tissue sections (8-10). Initially, ipsilateral SN was selected as a region of interest (ROI). Dopaminergic neurons were subsequently identified by automated detection of TH-labelled objects within this ROI, as previously validated by correlation analyses with traditional stereological methods (11, 12). Levels of total a-syn were assessed in this ROI by measuring the area and intensity of anti-a-syn staining.

Confocal images of primary cortical neurons, dopaminergic neurons, and LAMP1 staining within the SN were processed using Imaris software (Oxford instruments). Z stacks were projected to give a 3D reconstruction of the field of view and mean pixel intensity per GFP⁺ cell (or RFP⁺ cell for V1S/SV2 experiments) was calculated for a-syn or LAMP1 signal using the software's surfaces module. For LAMP1 staining within the SN, the number of LAMP1⁺ puncta within GFP⁺ cells was calculated for each field of view; 3 fields of view were imaged/animal.

### Catecholamine Quantification by HPLC

HPLC was performed as described (13). The investigator was blinded to experimental groups and treatment conditions. Brain sections were homogenized followed by centrifugation at 10,000×g for 20 minutes. Catecholamines were determined from the supernatant. Values of catecholamines are expressed as ng analyte/mg total protein.

### Statistical Analysis

All data are represented as mean±s.d. with at least 3 independent experiments, unless otherwise stated. Statistical analysis was performed using GraphPad Prism 8.

TABLE 2
Peptide counts from the screens. Illumina sequencing results after
demultiplexing, demonstrating peptide counts. Only reads with
an average quality Phred score of >30 were selected, and
frequencies were calculated. Reads were then normalized
to the total number of peptides read for that sample
population, and a scalar factor was applied.
Alpha Syn

| SEQ | | Reads | Reads | Reads | Reads | Slope | | |
|---|---|---|---|---|---|---|---|---|
| ID | | 0 uM | 10 UM | 25 UM | 50 UM | Norm | # Mapped | # PD GO |
| NO: | Peptide | MG132 | MG132 | MG132 | MG132 | Reads | Complexes | Terms |
| 1 | IPIQLKA | 90 | 1 | 1629 | 3375 | 3.45 | 11 | 16 |
| 49 | RAPSCHL | 1 | 13 | 33 | — | 0.15 | 2 | 50 |
| 50 | DWLMVNL | 1 | 13 | 29 | — | 0.13 | 3 | 5 |
| 51 | LPASMPC | 2 | 16 | 27 | — | 0.09 | 1 | 0 |
| 52 | LQVGVAV | 1 | 25 | 23 | — | 0.09 | 13 | 18 |
| 53 | PLPTGMG | 1 | 3 | 20 | — | 0.08 | 7 | 8 |
| 54 | CMVHSAG | 4 | 14 | 33 | — | 0.08 | 1 | 1 |
| 55 | AAYNLCA | 1 | 14 | 19 | — | 0.07 | 6 | 18 |
| 56 | LSGECVP | 3 | 28 | 28 | — | 0.07 | 9 | 27 |
| 57 | GAAAGGT | 1 | 10 | 17 | — | 0.06 | 70 | 162 |

Alpha syn A53T

| SEQ | | Reads | Reads | Reads | Reads | Slope | | |
|---|---|---|---|---|---|---|---|---|
| ID | | 0 uM | 10 UM | 25 UM | 50 UM | Norm | # Mapped | # PD GO |
| NO: | Peptide | MG132 | MG132 | MG132 | MG132 | Reads | Complexes | Terms |
| 1 | IPIQLKA | 168 | 20 | 3859 | 13308 | 40.54 | 11 | 16 |
| 58 | QSVVTAP | 8 | 6 | 37 | 465 | 1.38 | 16 | 30 |
| 59 | CKYRSVL | 5 | 7 | 11 | 438 | 1.30 | 4 | 9 |
| 60 | PAMQIDG | 32 | 20 | 53 | 337 | 0.96 | 6 | 4 |
| 61 | GWTTVRR | 15 | 1 | 29 | 322 | 0.94 | 0 | 0 |
| 62 | QSVLQQV | 13 | 2 | 12 | 282 | 0.82 | 8 | 37 |
| 63 | QSPSSAW | 9 | 45 | 59 | 284 | 0.82 | 19 | 46 |
| 64 | QVSVAQQ | 45 | 13 | 120 | 246 | 0.69 | 9 | 18 |
| 65 | QAELSNS | 6 | 8 | 10 | 235 | 0.69 | 5 | 39 |
| 66 | EDVNKCV | 6 | 6 | 13 | 233 | 0.68 | 4 | 11 |

Oligomers Screen

| SEQ | | Reads | Reads | Reads | | | |
|---|---|---|---|---|---|---|---|
| ID | | GFP | GFP | GFP | Reads | # Mapped | |
| NO: | Peptide | Neg | Inter | High | Ratio | Complexes | # PD GO Terms |
| 1 | IPIQLKA | 15556 | 2853 | 12 | 1,218.98 | 11 | 16 |
| 67 | WPRYPHI | 92 | 0 | 1 | 47.36 | 2 | 1 |
| 68 | CFLFQIQ | 88 | 0 | 1 | 45.32 | 0 | 0 |
| 69 | ECHTKIR | 76 | 0 | 1 | 39.21 | 2 | 9 |
| 70 | MQMKLSQ | 70 | 0 | 1 | 36.15 | 6 | 45 |
| 71 | TQKRELT | 69 | 0 | 1 | 35.64 | 7 | 21 |
| 72 | VYLDILG | 59 | 0 | 1 | 30.55 | 7 | 2 |
| 73 | LKRDQPV | 118 | 0 | 3 | 30.30 | 21 | 103 |
| 74 | NNQYSFV | 58 | 0 | 1 | 30.04 | 10 | 29 |
| 75 | LPSKIYK | 57 | 0 | 1 | 29.53 | 14 | 167 |

### Example 4

Peptides were purchased from LifeTein. Cyclic angio-peptides were synthesized as follow (N-Terminal: FITC-Ahx, C-Terminal: Amidation, Disulfide Bridge between cysteine residues). Non-cyclic angio-peptides were synthesized as follow (N-Terminal: FITC-Ahx, C-Terminal: Amidation). For the cyclic bisphenyl peptide, (N-Terminal: FITC-Ahx, C-Terminal: Amidation, Perfluoroarene-based stapling on both cysteine residues using Decafluorobiphenyl).

Description: Cell viability effect of peptides in A53T expressing HEK293 cells at different concentrations of cyclized and non-cyclized angio-conjugated peptides and cyclic bisphenyl conjugated peptides was tested. The results are shown in FIG. 16.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

1. C. Corbi-Verge, P. M. Kim, Motif mediated protein-protein interactions as drug targets. Cell Commun Signal 14, 8 (2016).
2. A. Mullard, Protein-protein interaction inhibitors get into the groove. Nat Rev Drug Discov 11, 173-175 (2012).
3. C. Corbi-Verge, M. Garton, S. Nim, P. M. Kim, Strategies to Develop Inhibitors of Motif-Mediated Protein-Protein Interactions as Drug Leads. Annu Rev Pharmacol Toxicol 57, 39-60 (2017).
4. T. L. Nero, C. J. Morton, J. K. Holien, J. Wielens, M. W. Parker, Oncogenic protein interfaces: small molecules, big challenges. Nat Rev Cancer 14, 248-262 (2014).
5. D. M. O'Hara, S. K. Kalia, L. V. Kalia, Emerging disease-modifying strategies targeting α-synuclein for the treatment of Parkinson's disease. Br J Pharmacol 175, 3080-3089 (2018).
6. L. V. Kalia, S. K. Kalia, P. J. McLean, A. M. Lozano, A. E. Lang, α-Synuclein oligomers and clinical implications for Parkinson disease. Ann Neurol 73, 155-169 (2013).
7. M. G. Spillantini et al., Alpha-synuclein in Lewy bodies. Nature 388, 839-840 (1997).
8. M. Xilouri, O. R. Brekk, L. Stefanis, Autophagy and Alpha-Synuclein: Relevance to Parkinson's Disease and Related Synucleopathies. Mov Disord 31, 178-192 (2016).
9. H. Cheruvara, V. L. Allen-Baume, N. M. Kad, J. M. Mason, Intracellular screening of a peptide library to derive a potent peptide inhibitor of a-synuclein aggregation. J Biol Chem 290, 7426-7435 (2015).
10. P. Tompa, N. E. Davey, T. J. Gibson, M. M. Babu, A million peptide motifs for the molecular biologist. Mol Cell 55, 161-169 (2014).
11. S. Garrido-Urbani et al., Proteomic peptide phage display uncovers novel interactions of the PDZ1-2 supramodule of syntenin. FEBS Lett 590, 3-12 (2016).
12. S. Nim et al., Pooled screening for antiproliferative inhibitors of protein-protein interactions. Nat Chem Biol 12, 275-281 (2016).
13. Y. Ivarsson et al., Large-scale interaction profiling of PDZ domains through proteomic peptide-phage display using human and viral phage peptidomes. Proc Natl Acad Sci USA 111, 2542-2547 (2014).
14. T. F. Outeiro et al., Formation of toxic oligomeric alpha-synuclein species in living cells. PLoS One 3, e1867 (2008).
15. M. Kiechle et al., In Vivo Protein Complementation Demonstrates Presynaptic α-Synuclein Oligomerization and Age-Dependent Accumulation of 8-16-mer Oligomer Species. Cell Rep 29, 2862-2874.e2869 (2019).
16. H. Dimant et al., Direct detection of alpha synuclein oligomers in vivo. Acta Neuropathol Commun 1, 6 (2013).
17. J. E. Tetzlaff et al., CHIP targets toxic alpha-Synuclein oligomers for degradation. J Biol Chem 283, 17962-17968 (2008).
18. M. Picillo et al., Parkinsonism due to A53E α-synuclein gene mutation: Clinical, genetic, epigenetic, and biochemical features. Mov Disord 33, 1950-1955 (2018).
19. L. V. Kalia et al., Ubiquitinylation of α-synuclein by carboxyl terminus Hsp70-interacting protein (CHIP) is regulated by Bcl-2-associated athanogene 5 (BAG5). PLoS One 6, e14695 (2011).
20. M. D. Stuchell-Brereton et al., ESCRT-III recognition by VPS4 ATPases. Nature 449, 740-744 (2007).
21. E. Morel et al., Phosphatidylinositol-3-phosphate regulates sorting and processing of amyloid precursor protein through the endosomal system. Nat Commun 4, 2250 (2013).
22. M. Vietri, M. Radulovic, H. Stenmark, The many functions of ESCRTs. Nat Rev Mol Cell Biol 21, 25-42 (2020).
23. G. Skibinski et al., Mutations in the endosomal ESCRTIII-complex subunit CHMP2B in frontotemporal dementia. Nat Genet 37, 806-808 (2005).
24. L. E. Cox et al., Mutations in CHMP2B in lower motor neuron predominant amyotrophic lateral sclerosis (ALS). PLoS One 5, e9872 (2010).
25. D. Chang et al., A meta-analysis of genome-wide association studies identifies 17 new Parkinson's disease risk loci. Nat Genet 49, 1511-1516 (2017).
26. E. L. Clayton et al., Frontotemporal dementia caused by CHMP2B mutation is characterised by neuronal lysosomal storage pathology. Acta Neuropathol 130, 511-523 (2015).
27. T. Kurashige et al., Localization of CHMP2B-immunoreactivity in the brainstem of Lewy body disease. Neuropathology 33, 237-245 (2013).
28. G. Effantin et al., ESCRT-III CHMP2A and CHMP3 form variable helical polymers in vitro and act synergistically during HIV-1 budding. Cell Microbiol 15, 213-226 (2013).
29. H. Urwin et al., Disruption of endocytic trafficking in frontotemporal dementia with CHMP2B mutations. Hum Mol Genet 19, 2228-2238 (2010).
30. B. Spencer et al., α-Synuclein interferes with the ESCRT-III complex contributing to the pathogenesis of Lewy body disease. Hum Mol Genet 25, 1100-1115 (2016).
31. M. D. Tuttle et al., Solid-state NMR structure of a pathogenic fibril of full-length human a-synuclein. Nat Struct Mol Biol 23, 409-415 (2016).
32. Y. Chu, H. Dodiya, P. Aebischer, C. W. Olanow, J. H. Kordower, Alterations in lysosomal and proteasomal markers in Parkinson's disease: relationship to alpha-synuclein inclusions. Neurobiol Dis 35, 385-398 (2009).
33. M. S. Pols, J. Klumperman, Trafficking and function of the tetraspanin CD63. Exp Cell Res 315, 1584-1592 (2009).
34. J. Moscat, M. T. Diaz-Meco, p62 at the crossroads of autophagy, apoptosis, and cancer. Cell 137, 1001-1004 (2009).
35. L. Stefanis et al., How is alpha-synuclein cleared from the cell?J Neurochem 150, 577-590 (2019).
36. G. K. Tofaris et al., Ubiquitin ligase Nedd4 promotes alpha-synuclein degradation by the endosomal-lysosomal pathway. Proc Natl Acad Sci USA 108, 17004-17009 (2011).
37. J. R. Mazzulli et al., Gaucher disease glucocerebrosidase and a-synuclein form a bidirectional pathogenic loop in synucleinopathies. Cell 146, 37-52 (2011).
38. J. C. Bridi, F. Hirth, Mechanisms of α-Synuclein Induced Synaptopathy in Parkinson's Disease. Front Neurosci 12, 80 (2018).
39. J. B. Koprich, L. V. Kalia, J. M. Brotchie, Animal models of a-synucleinopathy for Parkinson disease drug development. Nat Rev Neurosci 18, 515-529 (2017).
40. J. B. Koprich, T. H. Johnston, M. G. Reyes, X. Sun, J. M. Brotchie, Expression of human A53T alpha-synuclein in the rat substantia nigra using a novel AAV1/2 vector produces a rapidly evolving pathology with protein aggregation, dystrophic neurite architecture and nigrostriatal degeneration with potential to model the pathology of Parkinson's disease. Mol Neurodegener 5, 43 (2010).
41. J. B. Koprich et al., Progressive neurodegeneration or endogenous compensation in an animal model of Parkinson's disease produced by decreasing doses of alpha-synuclein. PLoS One 6, e17698 (2011).
42. C. McKinnon et al., Early-onset impairment of the ubiquitin-proteasome system in dopaminergic neurons caused by alpha-synuclein. Acta Neuropathol Commun 8, 17 (2020).
43. S. Duty, P. Jenner, Animal models of Parkinson's disease: a source of novel treatments and clues to the cause of the disease. Br J Pharmacol 164, 1357-1391 (2011).
44. A. B. Singleton et al., alpha-Synuclein locus triplication causes Parkinson's disease. Science 302, 841 (2003).
45. M. J. Devine et al., Parkinson's disease induced pluripotent stem cells with triplication of the a-synuclein locus. Nat Commun 2, 440 (2011).
46. P. Barbuti et al., Using High-Content Screening to Generate Single-Cell Gene-Corrected Patient-Derived iPS Clones Reveals Excess Alpha-Synuclein with Familial Parkinson's Disease Point Mutation A30P. Cells 9, (2020).
47. A. H. Laperle et al., iPSC modeling of young-onset Parkinson's disease reveals a molecular signature of disease and novel therapeutic candidates. Nat Med 26, 289-299 (2020).
48. https://www.abcam.com/lysosomal-intracellular-activity-assay-kit-ab234622.html 49. S. Brenner, The genetics of Caenorhabditis elegans. Genetics 77, 71-94 (1974).
50. T. Kuwahara et al., Familial Parkinson mutant alpha-synuclein causes dopamine neuron dysfunction in transgenic Caenorhabditis elegans. J Biol Chem 281, 334-340 (2006).
51. K. Fujisawa, J. L. Wrana, J. G. Culotti, The slit receptor EVA-1 coactivates a SAX-3/Robo mediated guidance signal in C. elegans. Science 317, 1934-1938 (2007).
52. T. Schallert, S. M. Fleming, J. L. Leasure, J. L. Tillerson, S. T. Bland, CNS plasticity and assessment of forelimb sensorimotor outcome in unilateral rat models of stroke, cortical ablation, parkinsonism and spinal cord injury. Neuropharmacology 39, 777-787 (2000).
53. M. X. Henderson et al., Spread of alpha-synuclein pathology through the brain connectome is modulated by selective vulnerability and predicted by network analysis. Nat Neurosci 22, 1248-1257 (2019).
54. A. Jais et al., PNOC(ARC) Neurons Promote Hyperphagia and Obesity upon High-Fat-Diet Feeding. Neuron 106, 1009-1025.e1010 (2020).
55. Z. He et al., Transmission of tauopathy strains is independent of their isoform composition. Nat Commun 11, 7 (2020).
56. D. M. O'Hara, M. Kapadia, S. Ping, S. K. Kalia, L. V. Kalia, Semi-Quantitative Determination of Dopaminergic Neuron Density in the Substantia Nigra of Rodent Models using Automated Image Analysis. J Vis Exp, (2021).
57. K. Okonechnikov, O. Golosova, M. Fursov, Unipro UGENE: a unified bioinformatics toolkit. Bioinformatics 28, 1166-1167 (2012).
58. M. A. Larkin et al., Clustal W and Clustal X version 2.0. Bioinformatics 23, 2947-2948 (2007).
59. E Monseillier et al., Polypeptides derived from a-Synuclein binding partners to prevent a-Synuclein fibrils interaction with and take up by cells. PloS ONE (2020), 15(8):e0237328. 54.
60. Colin M. Fadzen et al., Perfluoroarene-Based Peptide Macrocycles to Enhance Penetration Across the Blood-Brain Barrier. Journal of the American Chemical Society 2017 139 (44), 15628-15631.
61. McKinnon C, De Snoo M L, Gondard E, Neudorfer C, Chau H, Ngana S G, O'Hara D M, Brotchie J M, Koprich J B, Lozano A M, Kalia L V, Kalia S K. Early-onset impairment of the ubiquitin-proteasome system in dopaminergic neurons caused by α-synuclein. Acta Neuropathol Commun. 2020 Feb. 14; 8(1):17. doi: 10.1186/s40478-020-0894-0. PMID: 32059750; PMCID: PMC7023783.

1. A method of decreasing a-syn levels and/or decreasing a-syn toxicity in a cell, the method comprising contacting the cell with a charged multivesicular body protein 2B: a-synuclein (CHMP2B:a-syn) inhibitor.

2. The method of claim 1 wherein the CHMP2B: a-syn inhibitor comprises a peptide comprising or consisting of a) a MIT-Interacting Motif (MIM) sequence or a sequence with at least 50% sequence identity to said MIM sequence or b) an a-syn interaction sequence or a sequence with at least 50% sequence identity to said a-syn interaction sequence, wherein the peptide binds CHMP2B and inhibits CHMP2B-a-syn interaction by at least 50%, wherein the peptide optionally comprises one or more exogenous residues, optionally interspersed for example for one or more, optionally two, cysteines.

3. The method of claim 2, wherein the peptide of a) consists of a sequence of 5 to 25, 5 to 20, 4 to 15, 4 to 10, 7 to 20, 7 to 15 or 7 to 10 amino acids or the peptide of b) consists of a sequence 5 to 60, 5 to 50, 5 to 40, 5 to 30, 5 to 25, 5 to 20, 5 to 15, 5 to 10, 7 to 30, 7 to 25, 7 to 20, or 7 to 15 amino acids.

4. The method of any one of claims 1 to 3, wherein the peptide of a) is at least 5 amino acids or at least 7 amino acids in length and/or less than 20 amino acids in length and/or wherein the peptide of b) is at least 5 amino acids or at least 7 amino acids in length and less than 60 amino acids in length, preferably wherein the peptide is or is less than 12 amino acids, optionally is 9 amino acids, 10 amino acids, 11 amino acids or 12 amino acids.

5. The method of any one of claims 1 to 4, wherein the peptide comprises IPIQLKA, or a sequence with at least 50% sequence identity to IPIQLKA (SEQ ID NO: 1) that binds to CHMPB2 or inhibits CHMP2B-a-syn interaction.

6. The method of any one of claims 1 to 5, wherein the peptide comprises IERQLKA (SEQ ID NO: 16) (DPpep1.1), EIERQLKALG (SEQ ID NO: 17) (DPpep1.2), DEEIERQLKALG (SEQ ID NO: 6) (DPpep1.3), DEEIERQLDALG (SEQ ID NO: 18) (DPpep1.4), IPKQEKA (SEQ ID NO: 19) (DPpep1.5), EEDDDMKELENWAGSM (SEQ ID NO: 20) (CHMP4-MIM), DEELERRLKALK (SEQ ID NO: 21) (SUPER), EQDELSQRLARLRDQV (SEQ ID NO: 22) (1B-MIM), VPVKARPRQAELVAAS (SEQ ID NO: 23) (6-MIM2), EDQLSRRLAALR (SEQ ID NO: 3) (A1-MIM), DADLEERLKNLR (SEQ ID NO: 5) (2A-MIM), LEAMQSRLATLR (SEQ ID NO: 7) (3-MIM), FDDLSRRFEELK (SEQ ID NO: 13) (IST1-MIM), RNERQLKALG (SEQ ID NO: 24) (optim1), EEEIVRQLKALG (SEQ ID NO: 25) (optim 2), DIEIEFQLKALG (SEQ ID NO: 26) (optim 3), EIERQLKAQI (SEQ ID NO: 27) (optim 4), DEEYERQWKALG (SEQ ID NO: 28) (optim 5), DEAIERVLKALG (SEQ ID NO: 29) (optim 7) DDEIEVQLKALG (SEQ ID NO: 30) (optim.8), TLEIERQLKA (SEQ ID NO: 31) (optim9) or LEEIERQLKALG (SEQ ID NO: 32) (optim10).

7. The method of any one of claims 1 to 6, wherein the peptide comprises or is DEEIERQLKALG (SEQ ID NO: 6) (DPpep1.3).

8. The method of any one of claims 1 to 7, wherein the peptide has or lacks a positive charge.

9. The method of any one of claims 1 to 4, wherein the peptide comprises NEEGAPQEGILE (SEQ ID NO: 34), or fragment thereof that is that is at least 5 amino acids long or at least 7 amino acids long.

10. The method of any one of claims 1 to 9, wherein the peptide is a stapled peptide.

11. The method of claim 9, wherein the stapled peptide comprises a sequence selected from DEEYCRQWKALC (SEQ ID NO: 35), DEEICRQLDALC (SEQ ID NO: 37) or DIEICFQLKALC (SEQ ID NO: 36).

12. The method of any one of claims 1 to 11, wherein the a-syn is oligomerized a-syn.

13. The method of any one of claims 1 to 12 wherein the cell is a neural cell.

14. The method of any one of claims 1 to 13, wherein the cell is in vivo and the cell is contacted by administering the CHMP2B:a-syn inhibitor to a subject in need thereof.

15. The method of claim 14, wherein the subject in need thereof is a subject with a synucleinopathy.

16. The method of claim 15, wherein the synucleinopathy is Parkinson's disease (PD) multiple system atrophy (MSA), dementia with Lewy bodies (DLB), the Lewy body variant of Alzheimer's Disease (AD), neurodegeneration with brain iron accumulation, Parkinson's disease dementia (PDD), Alzheimer's disease and/or prodromal PD/DLB/MSA (e.g., REM sleep behaviour disorder, primary autonomic failure, MCI-LB or DLB-MCI).

17. The method of any one of claims 14 to 16, wherein the subject comprises a mutation in an a-syn gene.

18. The method of claim 17, wherein the mutation is a SNCA triplication.

19. A method of inhibiting neural degeneration, the method comprising administering to a subject in need thereof a charged multivesicular body protein 2B: a-synuclein (CHMP2B:a-syn) inhibitor.

20. The method of claim 19 wherein the CHMP2B: a-syn inhibitor comprises a peptide consisting of a 5 to 25 amino acids and comprising a MIT-Interacting Motif (MIM) sequence or a sequence with at least 50% sequence identity to said MIM sequence that binds CHMP2B or inhibits CHMP2B-a-syn interaction by at least 50%.

21. The method of any preceding claim wherein the peptide of a) consists of a sequence of 5 to 25, 5 to 20, 4 to 15, 4 to 10, 7 to 20, 7 to 15 or 7 to 10 amino acids or the peptide of b) consists of a sequence 5 to 60, 5 to 50, 5 to 40, 5 to 30, 5 to 25, 5 to 20, 5 to 15, 5 to 10, 7 to 30, 7 to 25, 7 to 20, or 7 to 15 amino acids.

22. The method of claim 19, wherein the peptide of a) is at least 5 amino acids or at least 7 amino acids in length and/or less than 20 amino acids in length and/or wherein the peptide of b) is at least 5 amino acids or at least 7 amino acids in length and less than 60 amino acids in length, preferably wherein the peptide is or is less than 12 amino acids, optionally is 9 amino acids, 10 amino acids, 11 amino acids or 12 amino acids.

23. The method of any one of claims 1 to 22, wherein the peptide comprises IPIQLKA (SEQ ID NO: 1), or a sequence with at least 50% sequence identity to IPIQLKA (SEQ ID NO: 1) and inhibits CHMP2B-a-syn interaction.

24. The method of any one of claims 1 to 23, wherein the peptide comprises IERQLKA (SEQ ID NO: 16) (DPpep1.1), EIERQLKALG (SEQ ID NO: 17) (DPpep1.2), DEEIERQLKALG (SEQ ID NO: 6) (DPpep1.3), DEEIERQLDALG (SEQ ID NO: 18) (DPpep1.4), IPKQEKA (SEQ ID NO: 19) (DPpep1.5), EEDDDMKELENWAGSM (SEQ ID NO: 20) (CHMP4-MIM), DEELERRLKALK (SEQ ID NO: 21) (SUPER), EQDELSQRLARLRDQV (SEQ ID NO: 22) (1B-MIM), VPVKARPRQAELVAAS (SEQ ID NO: 23) (6-MIM2), EDQLSRRLAALR (SEQ ID NO: 3) (A1-MIM), DADLEERLKNLR (SEQ ID NO: 5) (2A-MIM), LEAMQSRLATLR (SEQ ID NO: 7) (3-MIM), FDDLSRRFEELK (SEQ ID NO: 13) (IST1-MIM),) RNERQLKALG (SEQ ID NO: 24) (optim1), EEEIVRQLKALG (SEQ ID NO: 25) (optim 2), DIEIEFQLKALG (SEQ ID NO: 26) (optim 3), EIERQLKAQI (SEQ ID NO: 27) (optim 4), DEEYERQWKALG (SEQ ID NO: 28) (optim 5), DEAIERVLKALG (SEQ ID NO: 29) (optim 7) DDEIEVQLKALG (SEQ ID NO: 30) (optim.8), TLEIERQLKA (SEQ ID NO: 31) (optim9) or LEEIERQLKALG (SEQ ID NO: 32) (optim10).

25. The method of any one of claims 1 to 24, wherein the peptide comprises or is DEEIERQLKALG (SEQ ID NO: 6) (DPpep1.3).

26. The method of any one of claims 1 to 25, wherein the peptide has or lacks a C-terminal positive charge.

27. The method of any one of claims 19 to 22, wherein the peptide comprises NEEGAPQEGILE (SEQ ID NO: 34), or fragment thereof that is that is at least 5 amino acids long or at least 7 amino acids long.

28. The method of any one of claims 1 to 27, wherein the peptide is a stapled peptide.

29. The method of claim 28, wherein the stapled peptide comprises a sequence selected from DEEYCRQWKALC (SEQ ID NO: 35), DEEICRQLDALC (SEQ ID NO: 37) or DIEICFQLKALC (SEQ ID NO: 36).

30. The method of any one of claims 1 to 29, wherein the a-syn is oligomerized a-syn.

31. The method of any one of claims 1 to 30, wherein the subject in need thereof is a subject with a synucleinopathy.

32. The method of claim 31, wherein the synucleinopathy is Parkinson's disease (PD), amyotrophic lateral sclerosis (ALS) or frontotemporal dementia (FTD).

33. The method of any one of claims 1 to 32, wherein the subject comprises a mutation in a-syn gene.

34. The method of claim 33 wherein the mutation is SNCA triplication.

35. A peptide or a polypeptide comprising or consisting of a) a MIT-Interacting Motif (MIM) sequence or a sequence with at least 50% sequence identity to said MIM sequence or b) an a-syn interaction sequence or a sequence with at least 50% sequence identity to said a-syn interaction sequence, wherein the peptide inhibits CHMP2B-CHMP2B interaction or CHMP2B-a-syn interaction by at least 50%, wherein the peptide optionally comprises one or more exogenous residues, optionally interspersed for example for one or more, optionally two, cysteines.

36. The peptide or polypeptide of claim 35 wherein the polypeptide comprises a peptide consisting of a 5 to 25 amino acids and comprising a MIT-Interacting Motif (MIM) sequence or a sequence with at least 50% sequence identity to said MIM sequence that binds CHMP2B and/or inhibits CHMP2B-a-syn interaction by at least 50% or wherein the polypeptide comprises a peptide consisting of a 5 to 60 amino acids and comprising an a-syn interaction sequence or a sequence with at least 50% sequence identity to said a-syn interaction sequence that binds CHMP2B and/or inhibits CHMP2B-a-syn interaction by at least 50%.

37. The peptide or polypeptide of any one of claims 35 to 36, wherein the peptide a) consists of a sequence of 5 to 20, 4 to 15, 4 to 10, 7 to 20, 7 to 15 or 7 to 10 amino acids or the peptide of b) consists of a sequence 5 to 60, 5 to 50, 5 to 40, 5 to 30, 5 to 25, 5 to 20, 5 to 15, 5 to 10, 7 to 30, 7 to 25, 7 to 20, or 7 to 15 amino acids.

38. The peptide or polypeptide of any one of claims 35 to 37, wherein the peptide of a) is at least 5 amino acids or at least 7 amino acids in length and/or less than 20 amino acids in length and/or wherein the peptide of b) is at least 5 amino acids or at least 7 amino acids in length and less than 60 amino acids in length, preferably wherein the peptide is or is less than 12 amino acids, optionally is 9 amino acids, 10 amino acids, 11 amino acids or 12 amino acids.

39. The peptide or polypeptide of any one of claims 35 to 38, wherein the peptide comprises IPIQLKA (SEQ ID NO: 1), or a sequence with at least 50% sequence identity to IPIQLKA (SEQ ID NO: 1) that inhibits CHMP2B-a-syn interaction.

40. The peptide or polypeptide of any one of claims 35 to 39, wherein the peptide comprises IERQLKA (SEQ ID NO: 16) (DPpep1.1), EIERQLKALG (SEQ ID NO: 17) (DPpep1.2), DEEIERQLKALG (SEQ ID NO: 6) (DPpep1.3), DEEIERQLDALG (SEQ ID NO: 18) (DPpep1.4), IPKQEKA (SEQ ID NO: 19) (DPpep1.5), EEDDDMKELENWAGSM (SEQ ID NO: 20) (CHMP4-MIM), DEELERRLKALK (SEQ ID NO: 21) (SUPER), EQDELSQRLARLRDQV (SEQ ID NO: 22) (1B-MIM), VPVKARPRQAELVAAS (SEQ ID NO: 23) (6-MIM2), EDQLSRRLAALR (SEQ ID NO: 3) (A1-MIM), DADLEERLKNLR (SEQ ID NO: 5) (2A-MIM), LEAMQSRLATLR (SEQ ID NO: 7) (3-MIM), FDDLSRRFEELK (SEQ ID NO: 13) (IST1-MIM), RNERQLKALG (SEQ ID NO: 24) (optim1), EEEIVRQLKALG (SEQ ID NO: 25) (optim 2), DIEIEFQLKALG (SEQ ID NO: 26) (optim 3), EIERQLKAQI (SEQ ID NO: 27) (optim 4), DEEYERQWKALG (SEQ ID NO: 28) (optim 5), DEAIERVLKALG (SEQ ID NO: 29) (optim 7) DDEIEVQLKALG (SEQ ID NO: 30) (optim.8), TLEIERQLKA (SEQ ID NO: 31) (optim9) or LEEIERQLKALG (SEQ ID NO: 32) (optim10).

41. The peptide or polypeptide of any one of claims 35 to 40, wherein the peptide comprises or is DEEIERQLKALG (SEQ ID NO: 6) (DPpep1.3).

42. The peptide or polypeptide of any one of claims 35 to 41, wherein the peptide has or lacks a positive charge.

43. The polypeptide of any one of claims 35 to 38, wherein the peptide comprises NEEGAPQEGILE (SEQ ID NO: 34), or fragment thereof that is at least 5 amino acids long or at least 7 amino acids long.

44. The peptide or polypeptide of any one of claims 35 to 43, wherein the peptide is a stapled peptide.

45. The peptide or polypeptide of claim 44, wherein the stapled peptide comprises a sequence selected from DEEYCRQWKALC (SEQ ID NO: 35), DEEICRQLDALC (SEQ ID NO: 37) or DIEICFQLKALC (SEQ ID NO: 36).

46. A charged multivesicular body protein 2B: a-synuclein (CHMP2B:a-syn) inhibitor comprising a peptide comprising a) a MIT-Interacting Motif (MIM) sequence or a sequence with at least 50% sequence identity to said MIM sequence or b) an a-syn interaction sequence or a sequence with at least 50% sequence identity to said a-syn interaction sequence, wherein the peptide that binds CHMP2B or inhibits CHMP2B-a-syn interaction by at least 50%, wherein the peptide optionally comprises one or more exogenous residues, optionally interspersed for example for one or more, optionally two cysteines.

47. The CHMP2B:a-syn inhibitor of claim 46 wherein the CHMP2B: a-syn inhibitor comprises a peptide consisting of a 5 to 25 amino acids and comprising a MIT-Interacting Motif (MIM) sequence or a sequence with at least 50% sequence identity to said MIM sequence that binds CHMP2Bor inhibits CHMP2B-a-syn interaction by at least 50%.

48. The CHMP2B:a-syn inhibitor of claim 46 or 47, wherein the peptide of a) consists of a sequence of 5 to 20, 4 to 15, 4 to 10, 7 to 20, 7 to 15 or 7 to 10 amino acids or the peptide of b) consists of a sequence 5 to 60, 5 to 50, 5 to 40, 5 to 30, 5 to 25, 5 to 20, 5 to 15, 5 to 10, 7 to 30, 7 to 25, 7 to 20, or 7 to 15 amino acids.

49. The CHMP2B:a-syn inhibitor of any one of claims 46 to 48, wherein the peptide of a) is at least 5 amino acids or at least 7 amino acids in length and/or less than 20 amino acids in length and/or wherein the peptide of b) is at least 5 amino acids or at least 7 amino acids in length and less than 60 amino acids in length, preferably wherein the peptide is or is less than 12 amino acids, optionally is 9 amino acids, 10 amino acids, 11 amino acids or 12 amino acids.

50. The CHMP2B:a-syn inhibitor of any one of claims 46 to 49, wherein the peptide is or is less than 12 amino acids.

51. The CHMP2B:a-syn inhibitor of any one of claims 46 to 50, wherein the peptide comprises IPIQLKA (SEQ ID NO: 1), or a sequence with at least 50% sequence identity to IPIQLKA (SEQ ID NO: 1) that inhibits CHMP2B-a-syn interaction.

52. The CHMP2B:a-syn inhibitor of any one of claims 46 to 51, wherein the peptide comprises IERQLKA (SEQ ID NO: 16) (DPpep1.1), EIERQLKALG (SEQ ID NO: 17) (DPpep1.2), DEEIERQLKALG (SEQ ID NO: 6) (DPpep1.3), DEEIERQLDALG (SEQ ID NO: 18) (DPpep1.4), IPKQEKA (SEQ ID NO: 19) (DPpep1.5), EEDDDMKELENWAGSM (SEQ ID NO: 20) (CHMP4-MIM), DEELERRLKALK (SEQ ID NO: 21) (SUPER), EQDELSQRLARLRDQV (SEQ ID NO: 22) (1B-MIM), VPVKARPRQAELVAAS (SEQ ID NO: 23) (6-MIM2), EDQLSRRLAALR (SEQ ID NO: 3) (A1-MIM), DADLEERLKNLR (SEQ ID NO: 5) (2A-MIM), LEAMQSRLATLR (SEQ ID NO: 7) (3-MIM), FDDLSRRFEELK (SEQ ID NO: 13) (IST1-MIM), RNERQLKALG (SEQ ID NO: 24) (optim1), EEEIVRQLKALG (SEQ ID NO: 25) (optim 2), DIEIEFQLKALG (SEQ ID NO: 26) (optim 3), EIERQLKAQI (SEQ ID NO: 27) (optim 4), DEEYERQWKALG (SEQ ID NO: 28) (optim 5), DEAIERVLKALG (SEQ ID NO: 29) (optim 7) DDEIEVQLKALG (SEQ ID NO: 30) (optim.8), TLEIERQLKA (SEQ ID NO: 31) (optim9) or LEEIERQLKALG (SEQ ID NO: 32) (optim10).

53. The CHMP2B:a-syn inhibitor of any any one of claims 46 to 52, wherein the peptide comprises or is DEEIERQLKALG (SEQ ID NO: 6) (DPpep1.3).

54. The CHMP2B:a-syn inhibitor of any one of claims 46 to 53, wherein the peptide has or lacks a positive charge.

55. The CHMP2B:a-syn inhibitor pf any one of claims 46 to 50, wherein the peptide comprises NEEGAPQEGILE (SEQ ID NO: 34), or fragment thereof that is that is at least 5 amino acids long or at least 7 amino acids long.

56. The CHMP2B:a-syn inhibitor of any one of claims 46 to 55, wherein the peptide is a stapled peptide.

57. The CHMP2B:a-syn inhibitor of claim 44, wherein the stapled peptide comprises a sequence selected from DEEYCRQWKALC (SEQ ID NO: 35), DEEICRQLDALC (SEQ ID NO: 37) or DIEICFQLKALC (SEQ ID NO: 36).

58. A nucleic acid molecule comprising a polynucleotide sequence encoding the peptide or polypeptide of any one of claims 35 to 45.

59. A vector comprising a vector backbone and the nucleic acid molecule of claim 57.

60. The vector of claim 58 wherein the vector backbone is a lentivirus or an adeno associated virus.

61. A recombinant cell recombinantly expressing the peptide or polypeptide of any one of claims 35 to 45 or comprising the nucleic acid molecule of claim 57 or the vector of claim 58 or 59.

62. A composition comprising the peptide or polypeptide, CHMP2B:a-syn inhibitor, nucleic acid, vector or cell of any one of claim 35 or 60.
